SEQUENCE LISTING (23 sequences)

SEQ ID NO: 1 - DNA, 1235 bp, Homo sapiens
ggggggggggggcacttggcttcaaagctggctcttggaaattgagcggagagcgacgcggttgttgtagctgccgctgcggccgccgcggaataataagccgggatctaccatacccattgactaactatggaagattataccaaaatagagaaaattggagaaggtacctatggagttgtgtataagggtagacacaaaactacaggtcaagtggtagccatgaaaaaaatcagactagaaagtgaagaggaaggggttcctagtactgcaattcgggaaatttctctattaaaggaacttcgtcatccaaatatagtcagtcttcaggatgtgcttatgcaggattccaggttatatctcatctttgagtttctttccatggatctgaagaaatacttggattctatccctcctggtcagtacatggattcttcacttgttaagagttatttataccaaatcctacaggggattgtgttttgtcactctagaagagttcttcacagagacttaaaacctcaaaatctcttgattgatgacaaaggaacaattaaactggctgattttggccttgccagagcttttggaatacctatcagagtatatacacatgaggtagtaacactctggtacagatctccagaagtattgctggggtcagctcgttactcaactccagttgacatttggagtataggcaccatatttgctgaactagcaactaagaaaccacttttccatggggattcagaaattgatcaactcttcaggattttcagagctttgggcactcccaataatgaagtgtggccagaagtggaatctttacaggactataagaatacatttcccaaatggaaaccaggaagcctagcatcccatgtcaaaaacttggatgaaaatggcttggatttgctctcgaaaatgttaatctatgatccagccaaacgaatttctggcaaaatggcactgaatcatccatattttaatgatttggacaatcagattaagaagatgtagctttctgacaaaaagtttccatatgttatgtcaacagatagttgtgtttttattgttaactcttgtctatttttgtcttatatatatttctttgttatcaaacttcagctgtacttcgtcttctaatttcaaaaatataacttaaaaatgtaaatattctatatgaatttaaatataattctgtaaatgtgaaaaaaaaaaaaaaaaaaaaa

SEQ ID NO: 2 - protein, 297 aa, Homo sapiens
MEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 3 - DNA, 948 bp, Homo sapiens
ccattgactaactatggaagattataccaaaatagagaaaattggagaaggtacctatggagttgtgtataagggtagacacaaaactacaggtcaagtggtagccatgaaaaaaatcagactagaaagtgaagaggaaggggttcctagtactgcaattcgggaaatttctctattaaaggaacttcgtcatccaaatatagtcagtcttcaggatgtgcttatgcaggattccaggttatatctcatctttgagtttctttccatggatctgaagaaatacttggattctatccctcctggtcagtacatggattcttcacttgttaaggtagtaacactctggtacagatctccagaagtattgctggggtcagctcgttactcaactccagttgacatttggagtataggcaccatatttgctgaactagcaactaagaaaccacttttccatggggattcagaaattgatcaactcttcaggattttcagagctttgggcactcccaataatgaagtgtggccagaagtggaatctttacaggactataagaatacatttcccaaatggaaaccaggaagcctagcatcccatgtcaaaaacttggatgaaaatggcttggatttgctctcgaaaatgttaatctatgatccagccaaacgaatttctggcaaaatggcactgaatcatccatattttaatgatttggacaatcagattaagaagatgtagctttctgacaaaaagtttccatatgttatgtcaacagatagttgtgtttttattgttaactcttgtctatttttgtcttatatatatttctttgttatcaaacttcagctgtacttcgtcttctaatttcaaaaatataacttaaaaatgtaaatattctatatgaatttaaatataattctgtaaatgtgaaaaaaaaaaaaaaaaaaaaa

SEQ ID NO: 4 - protein, 240 aa, Homo sapiens
MEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 5 - DNA, 3053 bp, Homo sapiens
gagcgcggctggagtttgctgctgccgctgtgcagtttgttcaggggcttgtggtggtgagtccgagaggctgcgtgtgagagacgtgagaaggatcctgcactgaggaggtggaaagaagaggattgctcgaggaggcctggggtctgtgaggcagcggagctgggtgaaggctgcgggttccggcgaggcctgagctgtgctgtcgtcatgcctcaaacccgatcccaggcacaggctacaatcagttttccaaaaaggaagctgtctcgggcattgaacaaagctaaaaactccagtgatgccaaactagaaccaacaaatgtccaaaccgtaacctgttctcctcgtgtaaaagccctgcctctcagccccaggaaacgtctgggcgatgacaacctatgcaacactccccatttacctccttgttctccaccaaagcaaggcaagaaagagaatggtccccctcactcacatacacttaagggacgaagattggtatttgacaatcagctgacaattaagtctcctagcaaaagagaactagccaaagttcaccaaaacaaaatactttcttcagttagaaaaagtcaagagatcacaacaaattctgagcagagatgtccactgaagaaagaatctgcatgtgtgagactattcaagcaagaaggcacttgctaccagcaagcaaagctggtcctgaacacagctgtcccagatcggctgcctgccagggaaagggagatggatgtcatcaggaatttcttgagggaacacatctgtgggaaaaaagctggaagcctttacctttctggtgctcctggaactggaaaaactgcctgcttaagccggattctgcaagacctcaagaaggaactgaaaggctttaaaactatcatgctgaattgcatgtccttgaggactgcccaggctgtattcccagctattgctcaggagatttgtcaggaagaggtatccaggccagctgggaaggacatgatgaggaaattggaaaaacatatgactgcagagaagggccccatgattgtgttggtattggacgagatggatcaactggacagcaaaggccaggatgtattgtacacgctatttgaatggccatggctaagcaattctcacttggtgctgattggtattgctaataccctggatctcacagatagaattctacctaggcttcaagctagagaaaaatgtaagccacagctgttgaacttcccaccttataccagaaatcagatagtcactattttgcaagatcgacttaatcaggtatctagagatcaggttctggacaatgctgcagttcaattctgtgcccgcaaagtctctgctgtttcaggagatgttcgcaaagcactggatgtttgcaggagagctattgaaattgtagagtcagatgtcaaaagccagactattctcaaaccactgtctgaatgtaaatcaccttctgagcctctgattcccaagagggttggtcttattcacatatcccaagtcatctcagaagttgatggtaacaggatgaccttgagccaagaaggagcacaagattccttccctcttcagcagaagatcttggtttgctctttgatgctcttgatcaggcagttgaaaatcaaagaggtcactctggggaagttatatgaagcctacagtaaagtctgtcgcaaacagcaggtggcggctgtggaccagtcagagtgtttgtcactttcagggctcttggaagccaggggcattttaggattaaagagaaacaaggaaacccgtttgacaaaggtgtttttcaagattgaagagaaagaaatagaacatgctctgaaagataaagctttaattggaaatatcttagctactggattgccttaaattcttctcttacaccccacccgaaagtattcagctggcatttagagagctacagtcttcattttagtgctttacacattcgggcctgaaaacaaatatgaccttttttacttgaagccaatgaattttaatctatagattctttaatattagcacagaataatatctttgggtcttactatttttacccataaaagtgaccaggtagaccctttttaattacattcactacttctaccacttgtgtatctctagccaatgtgcttgcaagtgtacagatctgtgtagaggaatgtgtgtatatttacctcttcgtttgctcaaacatgagtgggtatttttttgtttgttttttttgttgttgttgtttttgaggcgcgtctcaccctgttgcccaggctggagtgcaatggcgcgttctctgctcactacagcacccgcttcccaggttgaagtgattctcttgcctcagcctcccgagtagctgggattacaggtgcccaccaccgcgcccagctaattttttaatttttagtagagacagggttttaccatgttggccaggctggtcttgaactcctgaccctcaagtgatctgcccaccttggcctccctaagtgctgggattataggcgtgagccaccatgctcagccattaaggtattttgttaagaactttaagtttagggtaagaagaatgaaaatgatccagaaaaatgcaagcaagtccacatggagatttggaggacactggttaaagaatttatttctttgtatagtatactatgttcatggtgcagatactacaacattgtggcattttagactcgttgagtttcttgggcactcccaagggcgttggggtcataaggagactataactctacagattgtgaatatatttattttcaagttgcattctttgtctttttaagcaatcagatttcaagagagctcaagctttcagaagtcaatgtgaaaattccttcctaggctgtcccacagtctttgctgcccttagatgaagccacttgtttcaagatgactactttggggttgggttttcatctaaacacatttttccagtcttattagataaattagtccatatggttggttaatcaagagccttctgggtttggtttggtggcattaaatgg

SEQ ID NO: 6 - protein, 560 aa, Homo sapiens
MPQTRSQAQATISFPKRKLSRALNKAKNSSDAKLEPTNVQTVTCSPRVKALPLSPRKRLGDDNLCNTPHLPPCSPPKQGKKENGPPHSHTLKGRRLVFDNQLTIKSPSKRELAKVHQNKILSSVRKSQEITTNSEQRCPLKKESACVRLFKQEGTCYQQAKLVLNTAVPDRLPAREREMDVIRNFLREHICGKKAGSLYLSGAPGTGKTACLSRILQDLKKELKGFKTIMLNCMSLRTAQAVFPAIAQEICQEEVSRPAGKDMMRKLEKHMTAEKGPMIVLVLDEMDQLDSKGQDVLYTLFEWPWLSNSHLVLIGIANTLDLTDRILPRLQAREKCKPQLLNFPPYTRNQIVTILQDRLNQVSRDQVLDNAAVQFCARKVSAVSGDVRKALDVCRRAIEIVESDVKSQTILKPLSECKSPSEPLIPKRVGLIHISQVISEVDGNRMTLSQEGAQDSFPLQQKILVCSLMLLIRQLKIKEVTLGKLYEAYSKVCRKQQVAAVDQSECLSLSGLLEARGILGLKRNKETRLTKVFFKIEEKEIEHALKDKALIGNILATGLP

SEQ ID NO: 7 - DNA, 627 bp, Homo sapiens
agtctccggcgagttgttgcctgggctggacgtggttttgtctgctgcgcccgctcttcgcgctctcgtttcattttctgcagcgcgccacgaggatggcccacaagcagatctactactcggacaagtacttcgacgaacactacgagtaccggcatgttatgttacccagagaactttccaaacaagtacctaaaactcatctgatgtctgaagaggagtggaggagacttggtgtccaacagagtctaggctgggttcattacatgattcatgagccagaaccacatattcttctctttagacgacctcttccaaaagatcaacaaaaatgaagtttatctggggatcgtcaaatctttttcaaatttaatgtatatgtgtatataaggtagtattcagtgaatacttgagaaatgtacaaatctttcatccatacctgtgcatgagctgtattcttcacagcaacagagctcagttaaatgcaactgcaagtaggttactgtaagatgtttaagataaaagttcttccagtcagtttttctcttaagtgcctgtttgagtttactgaaacagtttacttttgttcaataaagtttgtatgttgcatttaaaaaaaaaaaaaaa

SEQ ID NO: 8 - protein, 79 aa, Homo sapiens
MAHKQIYYSDKYFDEHYEYRHVMLPRELSKQVPKTHLMSEEEWRRLGVQQSLGWVHYMIHEPEPHILLFRRPLPKDQQK

SEQ ID NO: 9 - DNA, 4415 bp, Homo sapiens
atgaagactccctgtattcccagtcctaagcaagggagcaaaggcaggggacagagccctgctgctcaggtgaggggccccacgtggaacgcgccggcgcgggaggggcggcctggcgcaggtcatttgggacatctgcgggttggcgcatgcgcgcgaggtgcgcaggctcgcgcctttttcccttttctaagctttctgtgttacccccggttccgctgtcttttctgtctacagtttgcgatccccgcgtccaggatggagcagctgaacgaactggagctgctgatggagaagagtttttgggaggaggcggagctgccggcggagctatttcagaagaaagtggtagcttcctttccaagaacagttctgagcacaggaatggataaccggtacctggtgttggcagtcaatactgtacagaacaaagagggaaactgtgaaaagcgcctggtcatcactgcttcacagtcactagaaaataaagaactatgcatccttaggaatgactggtgttctgttccagtagagccaggagatatcattcatttggagggagactgcacatctgacacttggataatagataaagattttggatatttgattctgtatccagacatgctgatttctggcaccagcatagccagtagtattcgatgtatgagaagagctgtcctgagtgaaacttttaggagctctgatccagccacacgccaaatgctaattggtacggttctccatgaggtgtttcaaaaagccataaataatagctttgccccagaaaagctacaagaacttgcttttcaaacaattcaagaaataagacatttgaaggaaatgtaccgcttaaatctaagtcaagatgaaataaaacaagaagtagaggactatcttccttcgttttgtaaatgggcaggagatttcatgcataaaaacacttcgactgacttccctcagatgcagctctctctgccaagtgataatagtaaggataattcaacatgtaacattgaagtcgtgaaaccaatggatattgaagaaagcatttggtcccctaggtttggattgaaaggcaaaatagatgttacagttggtgtgaaaatacatcgagggtataaaacaaaatacaagataatgccgctggaacttaaaactggcaaagaatcaaattctattgaacaccgtagtcaggttgttctgtacactctactaagccaagagagaagagctgatccagaggctggcttgcttctctacctcaagactggtcagatgtaccctgtgcctgccaaccatctagataaaagagaattattaaagctaagaaaccagatggcattctcattgtttcaccgtattagcaaatctgctactagacagaagacacagcttgcttctttgccacaaataattgaggaagagaaaacttgtaaatattgttcacaaattggcaattgtgctctttatagcagagcagttgaacaacagatggattgtagttcagtcccaattgtgatgctgcccaaaatagaagaagaaacccagcatctgaagcaaacacacttagaatatttcagcctttggtgtctaatgttaaccctggagtcacaatcgaaggataataaaaagaatcaccaaaatatctggctaatgcctgcttcggaaatggagaagagtggcagttgcattggaaacctgattagaatggaacatgtaaagatagtttgtgatgggcaatatttacataatttccaatgtaaacatggtgccatacctgtcacaaatctaatggcaggtgacagagttattgtaagtggagaagaaaggtcactgtttgctttgtctagaggatatgtgaaggagattaacatgacaacagtaacttgtttattagacagaaacttgtcggtccttccagaatcaactttgttcagattagaccaagaagaaaaaaattgtgatatagataccccattaggaaatctttccaaattgatggaaaacacgtttgtcagcaaaaaacttcgagatttaattattgactttcgtgaacctcagtttatatcctaccttagttctgttcttccacatgatgcaaaggatacagttgcctgcattctaaagggtttgaataagcctcagaggcaagcgatgaaaaaggtacttctttcaaaagactacacactcatcgtgggtatgcctgggacaggaaaaacaactacgatatgtactctcgtaagaattctctacgcctgtggttttagcgttttgttgaccagctatacacactctgctgttgacaatattcttttgaagttagccaagtttaaaataggatttttgcgtttgggtcagattcagaaggttcatccagctatccagcaatttacagagcaagaaatttgcagatcaaagtccattaaatccttagctcttctagaagaactctacaatagtcaacttatagttgcaacaacatgtatgggaataaaccatccaatattttcccgtaaaatttttgatttttgtattgtggatgaagcctctcaaattagccaaccaatttgtctgggccccctttttttttcacggagatttgtgttagtgggggaccatcagcagcttcctcccctggtgctaaaccgtgaagcaagagctcttggcatgagtgaaagcttattcaagaggctggagcagaataagagtgctgttgtacagttaaccgtgcagtacagaatgaacagtaaaattatgtccttaagtaataagctgacctatgagggcaagctggagtgtggatcagacaaagtggccaatgcagtgataaacctacgtcactttaaagatgtgaagctggaactggaattttatgctgactattctgataatccttggttgatgggagtatttgaacccaacaatcctgtttgtttccttaatacagacaaggttccagcgccagaacaagttgaaaaaggtggtgtgagcaatgtaacagaagccaaactcatagttttcctaacctccatttttgttaaggctggatgcagtccctctgatattggtattattgcaccgtacaggcagcaattaaagatcatcaatgatttattggcacgttctattgggatggtcgaagttaatacagtagacaaataccaaggaagggacaaaagtattgtcctagtatcttttgttagaagtaataaggatggaactgttggtgaactcttgaaagattggcgacgtcttaatgttgctataaccagagccaaacataaactgattcttctggggtgtgtgccctcactaaattgctatcctcctttggagaagctgcttaatcatttaaactcagaaaaattaatcattgatcttccatcaagagaacatgaaagtctttgccacatattgggtgactttcaaagagaataaaacactatttcccttgccttttcatactagggcagtatctcctctagctagtgcccatacagaaaattctatcaccatacaaaatttaatgcagtatttatgttttaaagcacaggtgtaccgaaaactgtgaaaagtctgaatttatgggttctatgcatgcatttttgcctaacctagagaaagagtttgataaatttttaccagctttgaagatggattaacttttgactttgagctttaaacttttaagtcagacatttcaggactaatttgattttgtagatatcattgtaagaactttatttgaaagactgaataaagggatttgatttgttttcatcatttaagcacagtcttgtgatgatgagaacataagtgtgattcttttctgtattttgaggtccctaatccaaagcccattttgctaggattttttctgctatcagatgtgttttcactctaaacctagtcttttatgacatgaattgattacttcctgttaattttctatcctcccttactatcctccttttttgttttcagtattcagtatttcagtattctagagtagattttgatataaaagaaaataattcttacatcatcttttgcaacaaattttgttttctgaattgataataaatttaaaaagttgattcctattttcacatatgttcatatgcccctatgtttgggggtatcactcagttttcccttttttgtgtaaagatgttttgtaaaacaaaattgtctcaaagtgattatattatatatataaaaagtaacagattttaacaaaggttaaaagattcttggggtaacagattcttctggggcttggaaatcttccatttctcttgagggttttttttaatgagtgttaaatatgttaaaatttttatttctacctcatgtgtttttttaaattattacttgaagttttttatttaataaattttttctactaatgg

SEQ ID NO: 10 - protein, 1146 aa, Homo sapiens
MKTPCIPSPKQGSKGRGQSPAAQVRGPTWNAPAREGRPGAGHLGHLRVGACARGAQARAFFPFLSFLCYPRFRCLFCLQFAIPASRMEQLNELELLMEKSFWEEAELPAELFQKKVVASFPRTVLSTGMDNRYLVLAVNTVQNKEGNCEKRLVITASQSLENKELCILRNDWCSVPVEPGDIIHLEGDCTSDTWIIDKDFGYLILYPDMLISGTSIASSIRCMRRAVLSETFRSSDPATRQMLIGTVLHEVFQKAINNSFAPEKLQELAFQTIQEIRHLKEMYRLNLSQDEIKQEVEDYLPSFCKWAGDFMHKNTSTDFPQMQLSLPSDNSKDNSTCNIEVVKPMDIEESIWSPRFGLKGKIDVTVGVKIHRGYKTKYKIMPLELKTGKESNSIEHRSQVVLYTLLSQERRADPEAGLLLYLKTGQMYPVPANHLDKRELLKLRNQMAFSLFHRISKSATRQKTQLASLPQIIEEEKTCKYCSQIGNCALYSRAVEQQMDCSSVPIVMLPKIEEETQHLKQTHLEYFSLWCLMLTLESQSKDNKKNHQNIWLMPASEMEKSGSCIGNLIRMEHVKIVCDGQYLHNFQCKHGAIPVTNLMAGDRVIVSGEERSLFALSRGYVKEINMTTVTCLLDRNLSVLPESTLFRLDQEEKNCDIDTPLGNLSKLMENTFVSKKLRDLIIDFREPQFISYLSSVLPHDAKDTVACILKGLNKPQRQAMKKVLLSKDYTLIVGMPGTGKTTTICTLVRILYACGFSVLLTSYTHSAVDNILLKLAKFKIGFLRLGQIQKVHPAIQQFTEQEICRSKSIKSLALLEELYNSQLIVATTCMGINHPIFSRKIFDFCIVDEASQISQPICLGPLFFSRRFVLVGDHQQLPPLVLNREARALGMSESLFKRLEQNKSAVVQLTVQYRMNSKIMSLSNKLTYEGKLECGSDKVANAVINLRHFKDVKLELEFYADYSDNPWLMGVFEPNNPVCFLNTDKVPAPEQVEKGGVSNVTEAKLIVFLTSIFVKAGCSPSDIGIIAPYRQQLKIINDLLARSIGMVEVNTVDKYQGRDKSIVLVSFVRSNKDGTVGELLKDWRRLNVAITRAKHKLILLGCVPSLNCYPPLEKLLNHLNSEKLIIDLPSREHESLCHILGDFQRE

SEQ ID NO: 11 - DNA, 13 bp, Homo sapiens
  misc_feature (6)..(8): n is a, c, g, or t
ggtcannntgacc

SEQ ID NO: 12 - DNA, 20 bp, Artificial sequence
  CDC2 forward primer for PCR reaction
cccaaatggaaaccaggaag

SEQ ID NO: 13 - DNA, 20 bp, Artificial sequence
  CDC2 reverse primer for PCR reaction
cgtttggctggatcatagat

SEQ ID NO: 14 - DNA, 20 bp, Artificial sequence
  CDC6 forward primer for PCR reaction
ggaaacccgtttgacaaagg

SEQ ID NO: 15 - DNA, 24 bp, Artificial sequence
  CDC6 reverse primer for PCR reaction
cgggtggggtgtaagagaagaatt

SEQ ID NO: 16 - DNA, 20 bp, Artificial sequence
  CKS2 forward primer for PCR reaction
ctgatgtctgaagaggagtg

SEQ ID NO: 17 - DNA, 20 bp, Artificial sequence
  CKS2 reverse primer for PCR reaction
ggaagaggtcgtctaaagag

SEQ ID NO: 18 - DNA, 20 bp, Artificial sequence
  DNA2L forward primer for PCR reaction
ggatggaactgttggtgaac

SEQ ID NO: 19 - DNA, 20 bp, Artificial sequence
  DNA2L reverse primer for PCR reaction
atttagtgagggcacacacc

SEQ ID NO: 20 - DNA, 18 bp, Artificial sequence
  ERbeta forward primer for PCR reaction
tccatgcgcctggctaac

SEQ ID NO: 21 - DNA, 22 bp, Artificial sequence
  ERbeta reverse primer for PCR reaction
cagatgttccatgcccttgtta

SEQ ID NO: 22 - DNA, 24 bp, Artificial sequence
  Beta actin forward primer for PCR reaction
acccacactgtgcccatctacgag

SEQ ID NO: 23 - DNA, 26 bp, Artificial sequence
  Beta actin reverse primer for PCR reaction
tctccttaatgtcacgcacgatttcc

(19) United States
(12) Patent Application Publication
(10) Pub. No.: US 2010/0261165 A1
(43) Pub. Date: Oct. 14, 2010

(54) Methods, Compositions and Kits for Use in Prognosis, Characterization and Treatment of Cancer

(75) Inventors: Chin-Yo Lin, Singapore (SG); Edison T. Liu, Singapore (SG)

Correspondence Address: EDELL, SHAPIRO & FINNAN, LLC, 1901 RESEARCH BLVD., SUITE 400, ROCKVILLE, MD, 20850-3164, US

(73) Assignee: AGENCY FOR SCIENCE, TECHNOLOGY AND RESEARCH, (SG)

(21) Appl. No.: 12/093,214

(22) PCT Filed: Nov. 14, 2006
(86) PCT No.: PCT/SG06/00347

§ 371 Date: Feb. 24, 2009

(30) Foreign Application Priority Data
Nov. 14, 2005 (US) 11273184

(51) Int. Cl. C12Q 1/68; C40B 40/06

(52) U.S. Cl. 435/6; 506/16

(57) ABSTRACT

The present invention provides methods, compositions and kits for the prognosis, characterization and treatment of cancers. The methods, compositions and kits of the invention comprise a set of markers whose expression is correlated with estrogen receptor beta function. Therefore, the methods, compositions and kits of the invention find particular use in endocrine-related cancers such as breast cancers.

All documents cited herein are incorporated by reference in their entirety for all purposes.

### FIELD OF THE INVENTION

The present invention relates 46 molecular genetic markers for prognosis, characterization and treatment of cancer, in particular breast cancer. In particular, the present invention relates to gene expression profile-based signatures for prognosis, characterization and treatment of cancer.

### BACKGROUND OF THE INVENTION

Breast cancer is thought to be the leading cause of death in women between the ages of 40 and 55. An estimated one million women worldwide are thought to be afflicted with some stage of breast cancer. Men are not immune to breast cancer either. Statistics have shown that if detected early, the five-year breast cancer survival rate exceeds 95%. Thus, considerable emphasis has, been placed on early detection of cellular transformation and tumor formation in breast tissue.

A marker-based approach to tumor identification and characterization promises improved prognosis and diagnosis of breast cancers. Traditionally, breast cancer diagnosis has relied on histopathological examination of the tumor. In addition to diagnosis of cancers, histopathological examinations also provide information about prognosis and selection of treatment regimens. Clinical parameters such as tumor size, tumour grade, the age of the individual and lymph node metastasis may also aid in prognosis. In addition to histopathological examination, diagnosis and/or prognosis may be determined by imaging methods such as mammograms or other X-ray imaging methods. All these techniques suffer from the drawbacks of cost, subjective interpretation and health risks to the individual.

Breast cancer is a hormone-dependent cancer and knowledge of whether a tumor is positive or negative for the presence of estrogen receptors (e.g. estrogen receptor α, (ER-α)) is used for prognosis and patient selection for anti-hormonal therapy.

A second isoform of estrogen receptor (ER) called ER-β, may influence tumor progression in ways different from those mediated by the ER-α isoform (37).

Estrogens are involved in a number of vertebrate developmental and physiological processes and have been implicated in certain types of endocrine-related cancers (1-4). Hormone response in target tissues is mediated by nuclear receptors that function as ligand-dependent transcription factors. Receptor function is further modulated by post-translational modifications and interactions with other nuclear proteins. Originally, only one type of estrogen receptor (ER) was thought to be involved in hormone signaling. However, a second ER, termed ER-β was subsequently discovered, adding another dimension of complexity to the regulation of hormone response. The original receptor was renamed ER-α (5).

ER-α and ER-β share 55% identity in their ligand-binding domains and approximately 97% similarity in the DNA-binding domains (DBD). Both ERs bind estradiol with high affinity, but vary in their ability to bind other natural and synthetic ligands and the types of response elicited upon ligand binding (6-8). Reflecting the high degree of similarity in their DBDs, both receptors interact with the same conserved estrogen response element (ERE; 5′-GGTCAnnnTGACC-3′) (SEQ ID No: 11) as either homodimers or α/β heterodimers (9-11). Tissue-specific expression and co-expression of receptor subtypes suggest that ER homodimers and heterodimers may mediate distinct hormone responses (12-15). Moreover, the discovery of ERβ variants with different structural and functional characteristics and tissue distribution further highlighted the potential complexity of the interactions between ERs and the mechanisms by which estrogen response is modulated (16-20).

The predominant impact of estrogen receptor activation appears to be alterations in the transcriptional activity and expression profiles of target genes. A number of genes, including trefoil factor 1/pS2, cathepsin D, cyclin D1, c-Myc, and the progesterone receptor, are positively regulated by estrogen treatment (21). Transcriptional repression by ER has not been as well studied. However, using Serial Analysis of Gene Expression (SAGE) and DNA microarrays, many more estrogen-responsive genes, induced and repressed by the hormone, have been identified and characterized (22-29). Much of the work on gene expression has been focused on the role of ERα, but little is known about genes specifically targeted by ERβ or by α/β heterodimers. Recent microarray experiments using knock-out animals indicate that target tissues in wild-type controls exhibited an overall reduced transcriptional response to hormone treatment as compared to ERβ knockouts (30). Expression studies of osteosarcoma cells stably transfected with each receptor subtype suggest that ERα and ERβ share some common target genes, although each also appear to have distinct sets of downstream targets (31). Despite these efforts the exact transcriptional effects of ERα and ERβ remain obscure.

ERβ has also been shown to antagonize the growth of ERα-positive hormone-dependent cultured breast tumor cells. ERβ function is currently inferred from its transcript (real-time PCR) and protein levels (ligand-binding and immunohistochemical assays). However the association of ERβ expression with breast cancer prognosis has been difficult to establish using transcript and protein levels as measures of ERβ activity, as increased gene expression does not necessarily lead to increased gene function and subsequent alteration in tumor behavior in vivo.

### SUMMARY OF THE INVENTION

The present inventors have identified cell cycle and DNA replication genes which are suppressed by ERβ overexpression in ERα-positive breast tumor cells. Expression profiles of these genes and ERβ in ERα-positive tumors from individuals who received adjuvant tamoxifen therapy revealed statistically significant inverse correlation of four genes, CDC2, CDC6, CKS2 and DNA2L, with ERβ transcript levels. Moreover, the expression profiles of this expression, cassette (five genes, including ERβ are associated with tumor grade, 10-year disease recurrence, and patient survival. Therefore the in vivo behavior of these genes appears to be associated with ERβ function and is predictive of disease outcome in breast cancer individuals treated with adjuvant endocrine therapy.

High-density DNA microarrays were employed to generate genome-wide expression profiles of genes responsive to ERβ expression and estrogen treatment in ERα-positive breast tumor cells. The expression profiles provided insights into the interactions between ERα and ERβ in regulating hormone response, and the potential roles of putative ERβ-modulated genes in the biology of primary breast tumors.

Accordingly, the present invention provides means to correlate ERβ expression and function with tumor behavior, thereby enabling better prognosis of clinical outcomes and conseqnent reduction in morbidity and mortality from breast cancers. More specifically, the present invention provides a gene expression cassette that is associated with both ERβ expression level and its transcriptional effects on downstream-cell cycle and DNA replication genes. Also provided are pharmaceutical compositions for treating breast cancers, and methods, kits and reagents for predicting outcomes and treating breast tumors.

In a first aspect of the present invention, there is provided a method of assessing ER-β function, the method comprising:
(i) determining the level of at least one marker selected from the group consisting of CDC2; CDC6, DNA2L and CKS2; and
(ii) using the level of the at least one said marker as an indication of ER-β function.

In one embodiment, the method defined in the first aspect of the present invention may be used for: (i) assessing treatment efficacy or cancer progression or regression; (ii) identifying a compound useful for the treatment of cancer; or (iii) assessing the carcinogenic potential of a test compound.

In another embodiment, the method may comprise determining the level of the marker(s) in a patient sample. In a preferred embodiment, the sample may be ER-α positive.

In another embodiment, the sample may be derived from a patient suffering from cancer. The cancer may, for example, be breast cancer, prostate cancer or colon cancer. The sample may be obtained at one or more time points. Expression levels of the marker(s) may optionally be compared with a control sample. The control sample may be a sample derived from a person not having cancer (e.g. breast cancer, prostate cancer or colon cancer). One or more control samples may be employed.

In a second aspect of the present invention; there is provided a method for cancer prognosis, the method comprising:
(i) determining the level of at least one marker selected from the group consisting of CDC2, CDC6, DNA2L and CKS2 in an ER-α positive sample of a patient; and
(ii) using the level of the at least one said marker in making a disease prognosis for the patient.

In a third aspect of the present invention, there is provided a method of selecting a treatment for a cancer patient, the method comprising:
(i) determining the level of at least one marker selected from the group consisting of CDC2, CDC6, DNA2L and CKS2 in a ER-α positive sample of the patient; and
(ii) using the level of the at least one said marker in selecting an appropriate treatment for the patient.

In a fourth aspect of the present invention, there is provided a method of characterizing a cancerous cell or a cell suspected of being cancerous, the method comprising:
(i) determining the level of at least one marker selected from the group consisting of CDC2, CDC6, DNA2L and CKS2; and
(ii) using the level of the at least one marker in the characterization of the cell(s); wherein the cell(s) are ER-α positive.

In some embodiments of the methods described above, the level of at least two or three markers selected from the group consisting of CDC2, CDC6, DNA2L and CKS2 may be determined. In one embodiment, the level of CDC2, CDC6, DNA2L and CKS2 may be determined.

In some embodiments, the cancer may be selected from the group consisting of breast cancer, prostate cancer and colon cancer.

In another embodiment, the level of a further marker may be determined, i.e. a marker other than a CDC2, CDC6; DNA2L and CKS2. The further marker may optionally be selected from Table 4 or may be ER-β.

In another embodiment, the cancer may be treated with adjuvant endocrine therapy.

In a fifth aspect of the present invention, there is provided an array for assessing ER-β function, comprising probes for determining the level of at least one, two or three markers selected from the group consisting of CDC2, CDC6, DNA2L and CKS2.

In one embodiment, the array may comprise probes for determining the level of CDC2, CDC6, DNA2L and CKS2. In one embodiment, the array may comprise probes which are bound to nucleic acids or proteins derived from a sample (e.g. a cell) which is suspected of being cancerous or which is known to be cancerous. The derived nucleic acids or proteins are not necessarily physically, derived from the sample but may be obtained in any manner including, for example, DNA replication (e.g. by PCR). Hence a broad interpretation of the phrase “derived from” is intended. Thus, the array in “use”, i.e. an array that is bound to nucleic acids or proteins derived from a sample is encompassed.

It will be appreciated that one embodiment of the invention provides for the use of the array in the methods of the invention in determining the level of the marker(s).

In a sixth aspect of the present invention, there is provided a kit for assessing ER-β function, comprising a reagent for determining the level of at least one, two or three markers selected from the group consisting of CDC2, CDC6, DNA2L and CKS2. In some embodiments, reagents for determining the level of CDC2, CDC6, DNA2L and CKS2 may be provided.

Details of the aspects and embodiments of the invention described herein are set forth in the accompanying drawings and the description below. Other features, objects, and advantages of the invention will be apparent from the description and drawings, and from the claims.

### DEFINITIONS

The following words and phrases (and where appropriate grammatical variants thereof) may be defined as set forth below.

As used in this application, the singular form “a,” “an,” and “the” include plural references unless the context clearly dictates otherwise. For example, the term “an agent” includes a plurality of agents, including mixtures thereof.

An individual is not limited to a human being but may comprise other organisms including but not limited to vertebrates and invertebrates or one or more cells derived from the same. Examples of individuals include any animal, including rodents (e.g. mice, rats, guinea pigs, hamster), rabbits, cats, dogs, pigs, goats, sheep, monkeys (or other primates), horses, cows, etc. The individual may be male or female. The female may be pre or post menopausal. The individual may be a cancer patient. The individual may be a clinical patient, a clinical trial volunteer, an experimental animal etc. The individual may be in need of treatment, for example by surgery, therapy, diagnosis or prognosis etc. The individual may, for example, be suspected of suffering from cancer, or suspected of being predisposed to cancer, or may have previously suffered from cancer or may currently be suffering from cancer. In one embodiment the cancer is a primary cancer. In one embodiment the cancer is selected from the group consisting of breast cancer, prostate cancer and colon cancer. In one embodiment the cancer is primary breast cancer. In one embodiment the individual has received adjuvant endocrine therapy (and may optionally still be receiving adjuvant endocrine therapy) or has been identified as being a suitable patient for adjuvant endocrine therapy (e.g. on the basis of ER-α status). The terms individual and patient are used herein interchangeably.

As used herein, the term “comprising” means “including.” Variations of the word “comprising”, such as “comprise” and “comprises,” have correspondingly varied meanings. Thus, for example, a composition “comprising” X may consist exclusively of X or may include one or more additional components.

The term “antibody” means an immunoglobulin molecule able to bind to a specific epitope on an antigen. Antibodies can be comprised of a polyclonal mixture, or may be monoclonal in nature. Further, antibodies can be entire immunoglobulins derived from natural sources, or from recombinant sources. The antibodies of the present invention may exist in a variety of forms, including for example as a whole antibody, or as an antibody fragment, or other immunologically active fragment thereof, such as complementarity determining regions. Similarly, the antibody may exist as an antibody fragment having functional antigen-binding domains, that is, heavy and light chain variable domains. Also, the antibody fragment may exist in a form selected from the group consisting of, but not limited to: Fv, F_{ab}, F(ab)₂; scFv (single chain Fv), dAb (single domain antibody), bi-specific antibodies, diabodies and triabodies.

As used herein, an “array” includes an intentionally created collection of molecules (e.g. probes) which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports. The term “array” includes, inter alia, those libraries of nucleic acids which can be prepared by spotting nucleic acids of essentially any length (e.g., from 1 to about 1000 nucleotide monomers in length) onto a substrate. As used herein, the term array and microarray may be used interchangeably.

Throughout this disclosure, various, aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “complementary” refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%; and more preferably from about 98 to 100% of the nucleotides of the other strand. Alternatively, complementarity exists when an RNA or DNA strand will hybridize under selective hybridization conditions to its complement. Typically, selective hybridization will occur when there is at least about 65% complementarity over a stretch of at least 14 to 25 nucleotides, preferably at least about 75%, and more preferably at least about 90% complementarity.

As used herein, the term “determining” is intended to include measuring the expression level (e.g. the amount or concentration of) a marker (e.g. nucleic acid or protein) of the invention. The term thus refers to the use of the materials, compositions and methods of the present invention for qualitative, and quantitative determination. By determining the level of a particular marker, we include qualitative determination (e.g. comparing the level of the marker in a sample with the level of the marker of a control sample or with the level of the marker from a sample obtained from the same patient but at a different time point) or quantitative determination (e.g. measuring the amount or concentration) of the level of a nucleic acid or protein which is encoded by or which corresponds to the particular marker (marker nucleic acids and marker proteins, respectively). For example, detecting a change in expression levels may include quantifying a change of any value between 10% and 90%, or of any value between 30% and 60%, or over 100%, of a marker of the invention when compared to a control. In other embodiments, detecting an increase in gene expression levels may include quantifying a change of any value between 1 to 5 fold or more of any of the markers of the invention when compared to a control.

As used herein “Disease-specific survival” or DSS refers to a survival assessment where the end point being examined is death because of a specific disease, for example, breast cancer. DSS refers to survival from a particular disease for a defined period of time and is usually reported as time since diagnosis or treatment. Death from some other cause is not considered when calculating DSS.

As used herein, “Disease-free survival” or DFS refers to a survival assessment where the end points are either tumor recurrence (e.g., the cancer comes back as a consequence of distant metastasis, to other sites in the body) or death (for example, because of breast cancer without evidence of distant metastasis). The term “disease-free-survival” as used herein is defined as a time between diagnosis or surgery to treat a cancer patient and reoccurrence. For example, a disease-free survival is “low” if the cancer patient has a first reoccurrence within five years after tumor resection, and more specifically, if the cancer patient has less than about 55% disease-free survival over 5 years. For example, a high disease-free survival refers to at least about 55% disease-free survival over 5 years.

The term “endocrine therapy” as used herein is defined as a treatment of or pertaining to any of the ducts or endocrine glands characterized by secreting internally end into the bloodstream from the cells of the gland. The treatment may remove the gland, block hormone synthesis, or prevent the hormone from binding to its receptor. Non-limiting examples of endocrine therapies that are contemplated by the present invention include tamoxifen, raloxifene, or other SERMs (selective estrogen-receptor modulators). Tamoxifen has been the most commonly prescribed drug to treat breast cancer since its approval by the U.S. Food and Drug Administration (FDA) in the 1970s. Tamoxifen is an anti-estrogen and works by competing with the hormone estrogen to bind to estrogen receptors in breast cancer cells. Tamoxifen has been shown to reduce the risk of recurrence of an original cancer and the risk of developing new cancers by working against the effects of estrogen on breast cancer cells. A pharmaceutical composition comprising tamoxifen is generally administered as an oral composition such as a pill or capsule. Tamoxifen belongs to a class of agents known as selective estrogen receptor modulators. These agents display estrogen antagonist activity on some genes and agonist activity on others.

As used herein, a “fragment,” or “segment,” refers to a portion of a larger polynucleotide that is capable of being differentially expressed or detected in an assay or screening method according to the invention. A polynucleotide, for example, can be broken up, or fragmented into, a plurality of segments. Various methods of fragmenting nucleic acids are well known in the art. These methods may be, for example, either chemical or physical in nature. Chemical fragmentation may include partial degradation with a nuclease e.g. DNase; partial depurination with acid; the use of restriction enzymes; intron-encoded endonucleases; DNA-based cleavage methods, such as triplex and hybrid formation methods; that rely on the specific hybridization of a nucleic acid segment to localize a cleavage agent to a specific location in the nucleic-acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.”) which is incorporated herein by reference for all purposes. These methods can be optimized to digest a nucleic acid into fragments of a selected size range. Useful size ranges may be from 10, 15, 25, 50, 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

As used-herein, the term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.”The proportion of the population of polynucleotides that forms stable hybrids is referred to herein as the “degree of hybridization.”

Hybridization conditions will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different under different circumstances. Longer fragments may require higher hybridization temperatures for specific hybridization. As other factors may affect the stringency of hybridization, including base composition and length of the complementary strands, presence of organic solvents and extent of base mismatching, combination of parameters is more important than the absolute measure of any one alone. Generally, stringent conditions are selected to be about 5° C. lower than the thermal melting point (Tₘ) for the specific sequence at a defined ionic strength and pH. The Tₘ is the temperature (under defined ionic strength, pH and nucleic acid composition) at which 50% of the probes complementary to the target sequence hybridize to the target sequence at equilibrium. Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2nd Ed. Cold Spring Harbor press (1989) and Anderson “Nucleic Acid Hybridization” 1st Ed., BIOS Scientific Publishers Limited (1999), which are hereby incorporated by reference in their entireties for all purposes above.

The term “probe” refers to any molecule which is capable of selectively binding to a specifically intended target molecule, for example, a nucleotide transcript or protein. Probes can be either synthesized by one skilled in the art, or derived from appropriate biological preparations. For purposes of detection of the target molecule, probes may be specifically designed to be labeled. Examples of molecules that can be utilized as probes include, but are not limited to, RNA, DNA, proteins, antibodies, and organic molecules. In some embodiments, a probe can be surface immobilized. Where nucleic acids (such as oligonucleotides) are used they may be capable of binding in a base-specific manner to another strand of nucleic acid. Hybridization may occur between complementary nucleic acid strands or between nucleic acid strands that contain minor regions of mismatch. Such probes include peptide nucleic acids, as described in Nielsen et al., Science 254:1497-1500 (1991); Nielsen Curr. Opin. Biotechnol., 10:71-75 (1999) and other nucleic acid analogs and nucleic acid mimetics.

As used herein, “mRNA” includes, but is not limited to, pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

As used herein, the term “nucleic acid”, and equivalent terms such as polynucleotide, refers to a polymeric form of nucleotides of any length, such as ribonucleotides, deoxyribonucleotides or peptide nucleic acids (PNAs), that comprise purine and pyrimidine bases; or other natural, chemically or biochemically modified, non-natural, or derivatized nucleotide bases. The nucleic acid may be double stranded or single stranded. References to single stranded nucleic acids include references to the sense or antisense strands. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The terms nucleoside, nucleotide, deoxynucleoside and deoxynucleotide generally include complements, fragments and variants of the nucleoside, nucleotide, deoxynucleoside and deoxynucleotide, or analogs thereof.

A “variant” is a nucleic acid molecule that is a recognized variation of a nucleic acid molecule or expression product thereof. Splice variants may be determined for example by using computer programs, e.g., BLAST. Allelic variants have in general a high percent identity to the nucleic acid molecule of interest. “Single nucleotide polymorophism” (SNP) refers to a change in a single base as a result of a substitution, insertion or deletion. The change may be conservative (purine for purine) or non-conservative (purine to pyrimidine) and may or may not result in a change in an encoded amino acid.

Analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. Analogs in general retain the biological activities of the naturally-occurring molecules but may confer advantages such as longer lifespan or enhanced activity. The changes can be tailor made to stabilize or destabilize hybrid formation or enhance the specificity of hybridization with a complementary nucleic acid sequence as desired.

The term “labeled”, with regard to, for example, a probe, is intended to encompass direct labeling of the probe by coupling (i.e., physically linking) a detectable substance to the probe, as well as indirect labeling of the probe by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

An “oligonucleotide” as used herein is a single stranded molecule which may be used in hybridization or amplification technologies. In general, an oligonucleotide may be any integer from about 15 to about 100 nucleotides in length, but may also be of greater length.

As used herein, “solid support”, “support”, and “substrate” are used interchangeably and include a reference to a material or group of materials which may have a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) may take the form of beads, resins, gels, microspheres, or other, geometric configurations. Examples of supports include glass, polystyrene, polypropylene, polyethylene, dextran, nylon, amylases, natural and modified celluloses, polyacrylamides, gabbros, and magnetite. See also U.S. Pat. No. 5,744,305 for exemplary substrates.

As used herein, the term “sample” includes tissues, cells, body fluids and isolates thereof etc., isolated from a subject, as well as tissues, cells and fluids etc. present within a subject (i.e. the sample is in vivo). Examples of samples include: blood fluids, lymph and cystic fluids, as well as nipple aspirates, sections of tissues such as biopsy and autopsy samples, frozen sections taken for histologic purposes, archival samples, blood, plasma, serum, sputum, stool, tears, mucus, hair, skin, ascitic fluid, cystic fluid, urine, nipple exudate, explants and primary and/or transformed cell cultures derived from patient tissues etc. In one embodiment the sample may be a “breast-associated” body fluid, which is a fluid which, when in the body of a patient, contacts or passes through breast cells, or into which cells, nucleic acids or proteins shed from breast cells are capable of passing. Examples of breast-associated body fluids include blood fluids, lymph, cystic fluid, and nipple aspirates.

As used herein the term “treatment”, refers to any and all methods which remedy a disease state or symptoms, prevent the establishment of disease, or otherwise prevent, hinder, retard, or reverse the progression of disease or other undesirable symptoms in any way whatsoever. The term “treatment” includes, inter alia: (i) the prevention or inhibition of cancer or cancer recurrence, (ii) the reduction or elimination of symptoms or cancer cells, and (iii) the substantial or complete elimination of the cancer in question. Treatment may be effected prophylactically or therapeutically. Treatment may entail treatment with a single agent or with a combination (more than two) of agents. An “agent” is used herein broadly to refer to, for example, a compound or other means for treatment e.g. radiation treatment or surgery.

Unless otherwise indicated, all technical and scientific terms used herein have the same meaning as commonly understood to one of ordinary skill in the art to which the invention belongs.

### BRIEF DESCRIPTION OF THE DRAWINGS

The file of this patent contains at least one drawing executed in color. Copies of this patent with color drawings will be provided by the Patent Office upon request and payment of the necessary fee.

The accompanying drawings, which are incorporated in and form a part of this specification, illustrate embodiments of the invention and, together with the description, serve to explain the disclosed principles of the invention.

FIG. 1. Similar expression profiles of responsive genes under all induction and treatment conditions. (A) 1847 genes up-regulated by ERβ expression (α+β; left panel), E2 treatment (α, +E2; center panel), or both in combination (α+β, +E2; right panel), as determined by fold-change ratios over untreated-uninduced controls (see Materials and Methods), were visualized by hierarchical clustering and the Eisen TreeView software. The columns represent time points arranged in chronological order, and each row represents the expression profile of a particular gene. By convention, up-regulated genes are indicated by red signals and down-regulated genes are indicated by green. The magnitude of change is proportional to the brightness of the signal. (B) Expression profiles of 1662 down-regulated responsive genes. Expression profiles of cell cycle and DNA replication genes differentially regulated by ERβ expression are highlighted by magenta colored boxes.

FIG. 2. Models of ligand-dependent interaction between ERα and ERβ. Differential regulation of gene expression by ERβ, in the presence of hormone ligand (E2+ERβ), as compared to ERα alone (E2). Responsive genes are divided into synergistic (A), attenuation (B), and antagonistic (C) models of interaction between receptor subtypes. Synergistic genes were defined by an increase in the magnitude of change in the same direction when ERβ was expressed and cells treated with E2 as compared to E2 treatment alone. The attenuation model includes genes whose expression levels were diminished by ERβ expression. In the antagonistic model, genes responded in opposite directions in ERβ expressing cells treated with E2 as compared to the hormone treated controls.

FIG. 3. Validation of estrogen-responsive regulation of cell cycle and DNA replication genes suppressed by ERβ over-expression by real-time quantitative PCR. CDC2, CDC6, DNA2L and CKS2 were selected for further validation based on their significant correlation with ERβ transcript levels and β actin expression was assessed as a negative control. Transcript levels in T-47 Dbeta cells were measured at 30 hours following estrogen treatment (+E2) or mock treatment and under induction conditions [−TET (+ERβ)] and non-induction (+TET) conditions. Relative fold-changes were calculated using the non-induced (+TET) samples as the reference.

FIG. 4. Clustering of 69 tumor samples by ERβ/ESR2, CDC2, CDC6, DNA2L and CKS2 expression profiles is associated with clinical parameters and disease outcome. (A) Hierarchical clustering of tumor samples into two groups of high (black dendrogram; Cluster 1) and low (red dendrogram; Cluster 2) expression clusters. Clinical parameters, including relapse and death from breast cancer within 10 years of surgery, and lymph node positive status are indicated with a solid bar beneath each tumor sample. Tumor grade is indicated by colored bars with green, blue, and red bars denoting grades 1, 2, and 3 respectively. Gray bars denote missing data. Significant distribution of clinical parameters between the two clusters was determined by Chi-square tests and P-values derived from the calculated distributions. Kaplan-Meier plots of (B) DFS and (C) DSS curves for Cluster 1 (black) and Cluster 2 (red) patients are shown with the associated P-values from likelihood-ratio analysis.

FIG. 5. Clustering of 45 previously published ERα-positive tumor samples (36) using the available genes from the ERβ expression cassette expression profile is associated with disease outcome. (A) Hierarchical clustering of tumor samples into two groups of high (black dendrogram) and low (red dendrogram) expression clusters. Kaplan-Meier plots of (B) DFS and (C) DSS curves for high (black) and low (red) expression patients are shown with the associated P-values. ERβ levels were not assessed due to the absence of ERβ probes on the arrays used by Sotiriou et al. (36).

### DETAILED DESCRIPTION OF THE INVENTION

When a patent, application, or other reference is cited or repeated below, it should be understood that it is incorporated by reference in its entirety for all purposes as well as for the proposition that is recited.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al: (2002) Biochemistry, 5^{th} Ed., W.H. Freeman Pub., New York, N.Y., all of which are herein incorporated in their entirety by reference for all purposes.

The present inventors have discovered a transcriptionally-regulated mechanism for the growth inhibitory effects of ERβ in ERα-positive breast tumor cells. The functional characterization of the downstream targets of ER-β transcriptional regulation provides strong evidence for a functional and beneficial impact of ER-β on the in vivo behavior of primary breast cancers.

In the present invention, the level of at least one, two or three markers selected from the group consisting of CDC2, CDC6, DNA2L and CKS2 is determined.

In one embodiment the level of at least CDC2 and CDC6; CDC2 and DNA2L; CDC2 and CKS2; CDC6 and DNA2L; CDC6 and CKS2; or DNA2L and CKS2 is determined. In one embodiment the level of at least CDC2, CDC6, and DNA2L; CDC2, CDC6, and CKS2; CDC2, DNA2L and CKS2; or CDC6, DNA2L and CKS2 is determined.

In one embodiment, the level of one, two, three or all four of CDC2, CDC6, DNA2L and CKS2 is determined.

For the avoidance of doubt, a reference to “a marker of the invention” or “one or more of the markers of the invention” etc. includes a reference to one, two, three or all four of CDC2, CDC6, DNA2L and CKS2 and optionally also ER-β. It also includes a reference to a “marker protein” and a “marker nucleic acid.”

The markers of the invention may be identified by their Accession numbers. Accession numbers, as used herein, may refer to Accession numbers from multiple databases, including GenBank, the European Molecular Biology Laboratory (EMBL), the DNA Database of Japan (DDBJ), or the Genome Sequence Database (GSDB), for nucleotide sequences, and including the Protein Information Resource. (PIR), SWISSPROT, Protein Research Foundation (PRF), and Protein Data Bank (PDB) (sequences from solved structures), as well as from translations from annotated coding regions from nucleotide sequences in GenBank, EMBL, DDBJ, or RefSeq, for polypeptide sequences. Accession numbers as used herein, may also refer to Accession numbers from databases such as UniGene, OMIM, LocusLink, or HomologoGene.

The sequences of the accession numbers are hereby expressly incorporated by reference. GenBank is available, see, e.g., Benson, et al. (1998) Nuc. Acids Res. 26:1-7; and http://www.ncbi.nhn.nih.gov/. Sequences are also available in other databases, 25 e.g., European Molecular Biology Laboratory (EMBL) and DNA Database of Japan (DDBJ).

As indicated in Table 1 below, reference to CDC2, CDC6, DNA2L, CKS2, and ER-β includes allelic variants of CDC2, CDC6, DNA2L, CKS2 and ER-β, complements of CDC2, CDC6, DNA2L, CKS2 and ER-β, as well as to the non-human counterparts (i.e. homologs) of CDC2, CDC6, DNA2L, CKS2 and ER-β. References to other markers are intended to be construed in a likewise manner.

TABLE 1

| | Human | Human | | | | | |
|---|---|---|---|---|---|---|---|
| Gene | Variant 1 | Variant 2 | Chimpanzee | Mouse | Rat | Fruit Fly | Worm |
| ER-β | NM_001437 | | | NM_207707 | NM_012754 | | |
| CDC2 | NM_001786 | NM_033379 | XM_507809 | NM_007659 | NM_019296 | NM_057449 | NM_171224 |
| | (SEQ ID NO. 1) | (SEQ ID NO. 3) | | | | | |
| | NP_001777 | NP_203698 | | | | | |
| | (SEQ ID NO. 2) | (SEQ ID NO. 4) | | | | | |
| CDC6 | NM_001254 | | XM_523625 | NM_011799 | XM_340896 | | NM_058942 |
| | (SEQ ID NO. 5) | | | | | | |
| | NP_001245 | | | | | | |
| | (SEQ ID NO. 6) | | | | | | |
| CKS2 | NM_001827 | | XM_520119 | NM_025415 | | NM_141560 | |
| | (SEQ ID NO. 7) | | | | | | |
| | NP_001818 | | | | | | |
| | (SEQ ID NO. 8) | | | | | | |
| DNA2L | XM_166103 | | | NM_177372 | XM_241671 | NM_132369 | NM_064115 |
| | (SEQ ID NO. 9) | | | | | | |
| | XP_166103 | | | | | | |
| | (SEQ ID NO. 10) | | | | | | |

The sequences of SEQ ID Nos. 1-10 are shown below:

SEQ ID NO. 1

>gi|16306490|ref|NM_001786.2| Homo sapiens cell
division cycle 2, G1 to S and G2 to M (CDC2),
transcript variant 1, mRNA
GGGGGGGGGGGGCACTTGGCTTCAAAGCTGGCTCTTGGAAATTGAGCGGA
GAGCGACGCGGTTGTTGTAGCTGCCGCTGCGGCCGCCGCGGAATAATAAG
CCGGGATCTACCATACCCATTGACTAACTATGGAAGATTATACCAAAATA
GAGAAAATTGGAGAAGGTACCTATGGAGTTGTGTATAAGGGTAGACACAA
AACTACAGGTCAAGTGGTAGCCATGAAAAAAATCAGACTAGAAAGTGAAG
AGGAAGGGGTTCCTAGTACTGCAATTCGGGAAATTTCTCTATTAAAGGAA
CTTCGTCATCCAAATATAGTCAGTCTTCAGGATGTGCTTATGCAGGATTC
CAGGTTATATCTCATCTTTGAGTTTCTTTCCATGGATCTGAAGAAATACT
TGGATTCTATCCCTCCTGGTCAGTACATGGATTCTTCACTTGTTAAGAGT
TATTTATACCAAATCCTACAGGGGATTGTGTTTTGTCACTCTAGAAGAGT
TCTTCACAGAGACTTAAAACCTCAAAATCTCTTGATTGATGACAAAGGAA
CAATTAAACTGGCTGATTTTGGCCTTGCCAGAGCTTTTGGAATACCTATC
AGAGTATATACACATGAGGTAGTAACACTCTGGTACAGATCTCCAGAAGT
ATTGCTGGGGTCAGCTCGTTACTCAACTCCAGTTGACATTTGGAGTATAG
GCACCATATTTGCTGAACTAGCAACTAAGAAACCACTTTTCCATGGGGAT
TCAGAAATTGATCAACTCTTCAGGATTTTCAGAGCTTTGGGCACTCCCAA
TAATGAAGTGTGGCCAGAAGTGGAATCTTTACAGGACTATAAGAATACAT
TTCCCAAATGGAAACCAGGAAGCCTAGCATCCCATGTCAAAAACTTGGAT
GAAAATGGCTTGGATTTGCTCTCGAAAATGTTAATCTATGATCCAGCCAA
ACGAATTTCTGGCAAAATGGCACTGAATCATCCATATTTTAATGATTTGG
ACAATCAGATTAAGAAGATGTAGCTTTCTGACAAAAAGTTTCCATATGTT
ATGTCAACAGATAGTTGTGTTTTTATTGTTAACTCTTGTCTATTTTTGTC
TTATATATATTTCTTTGTTATCAAACTTCAGCTGTACTTCGTCTTCTAAT
TTCAAAAATATAACTTAAAAATGTAAATATTCTATATGAATTTAAATATA
ATTCTGTAAATGTGAAAAAAAAAAAAAAAAAAAAA

SEQ ID NO. 2

>gi|4502709|ref|NP_001777.1| cell division cycle
2 protein isoform 1 [Homo sapiens]
MEDYTKIEKIGEGTYGVVYKGRHKTTGQVVAMKKIRLESEEEGVPSTAIR
EISLLKELRHPNIVSLQDVLMQDSRLYLIFEFLSMDLKKYLDSIPPGQYM
DSSLVKSYLYQILQGIVFCHSRRVLHRDLKPQNLLIDDKGTIKLADFGLA
RAFGIPIRVYTHEVVTLWYRSPEVLLGSARYSTPVDIWSIGTIFAELATK
KPLFHGDSEIDQLFRIFRALGTPNNEVWPEVESLQDYKNTFPKWKPGSLA
SHVKNLDENGLDLLSKMLIYDPAKRISGKMALNHPYFNDLDNQIKKM

SEQ ID NO. 3

>gi|27886643|ref|NM_033379.2| Homo sapiens cell
division cycle 2, G1 to S and G2 to M (CDC2),
transcript variant 2, mRNA
CCATTGACTAACTATGGAAGATTATACCAAAATAGAGAAAATTGGAGAAG
GTACCTATGGAGTTGTGTATAAGGGTAGACACAAAACTACAGGTCAAGTG
GTAGCCATGAAAAAAATCAGACTAGAAAGTGAAGAGGAAGGGGTTCCTAG
TACTGCAATTCGGGAAATTTCTCTATTAAAGGAACTTCGTCATCCAAATA
TAGTCAGTCTTCAGGATGTGCTTATGCAGGATTCCAGGTTATATCTCATC
TTTGAGTTTCTTTCCATGGATCTGAAGAAATACTTGGATTCTATCCCTCC
TGGTCAGTACATGGATTCTTCACTTGTTAAGGTAGTAACACTCTGGTACA
GATCTCCAGAAGTATTGCTGGGGTCAGCTCGTTACTCAACTCCAGTTGAC
ATTTGGAGTATAGGCACCATATTTGCTGAACTAGCAACTAAGAAACCACT
TTTCCATGGGGATTCAGAAATTGATCAACTCTTCAGGATTTTCAGAGCTT
TGGGCACTCCCAATAATGAAGTGTGGCCAGAAGTGGAATCTTTACAGGAC
TATAAGAATACATTTCCCAAATGGAAACCAGGAAGCCTAGCATCCCATGT
CAAAAACTTGGATGAAAATGGCTTGGATTTGCTCTCGAAAATGTTAATCT
ATGATCCAGCCAAACGAATTTCTGGCAAAATGGCACTGAATCATCCATAT
TTTAATGATTTGGACAATCAGATTAAGAAGATGTAGCTTTCTGACAAAAA
GTTTCCATATGTTATGTCAACAGATAGTTGTGTTTTTATTGTTAACTCTT
GTCTATTTTTGTCTTATATATATTTCTTTGTTATCAAACTTCAGCTGTAC
TTCGTCTTCTAATTTCAAAAATATAACTTAAAAATGTAAATATTCTATAT
GAATTTAAATATAATTCTGTAAATGTGAAAAAAAAAAAAAAAAAAAAA

SEQ ID NO. 4

>gi|16306492|ref|NP_203698.1| cell division cycle
2 protein isoform 2 [Homo sapiens]
MEDYTKIEKIGEGTYGVVYKGRHKTTGQVVAMKKIRLESEEEGVPSTAIR
EISLLKELRHPNIVSLQDVLMQDSRLYLIFEFLSMDLKKYLDSIPPGQYM
DSSLVKVVTLWYRSPEVLLGSARYSTPVDIWSIGTIFAELATKKPLFHGD
SEIDQLFRIFRALGTPNNEVWPEVESLQDYKNTFPKWKPGSLASHVKNLD
ENGLDLLSKMLIYDPAKRISGKMALNHPYFNDLDNQIKKM

SEQ ID NO. 5

>gi|51944959|ref|NM_001254.3| Homo sapiens CDC6
cell division cycle 6 homolog (S. cerevisiae)
(CDC6), mRNA
GAGCGCGGCTGGAGTTTGCTGCTGCCGCTGTGCAGTTTGTTCAGGGGCTT
GTGGTGGTGAGTCCGAGAGGCTGCGTGTGAGAGACGTGAGAAGGATCCTG
CACTGAGGAGGTGGAAAGAAGAGGATTGCTCGAGGAGGCCTGGGGTCTGT
GAGGCAGCGGAGCTGGGTGAAGGCTGCGGGTTCCGGCGAGGCCTGAGCTG
TGCTGTCGTCATGCCTCAAACCCGATCCCAGGCACAGGCTACAATCAGTT
TTCCAAAAAGGAAGCTGTCTCGGGCATTGAACAAAGCTAAAAACTCCAGT
GATGCCAAACTAGAACCAACAAATGTCCAAACCGTAACCTGTTCTCCTCG
TGTAAAAGCCCTGCCTCTCAGCCCCAGGAAACGTCTGGGCGATGACAACC
TATGCAACACTCCCCATTTACCTCCTTGTTCTCCACCAAAGCAAGGCAAG
AAAGAGAATGGTCCCCCTCACTCACATACACTTAAGGGACGAAGATTGGT
ATTTGACAATCAGCTGACAATTAAGTCTCCTAGCAAAAGAGAACTAGCCA
AAGTTCACCAAAACAAAATACTTTCTTCAGTTAGAAAAAGTCAAGAGATC
ACAACAAATTCTGAGCAGAGATGTCCACTGAAGAAAGAATCTGCATGTGT
GAGACTATTCAAGCAAGAAGGCACTTGCTACCAGCAAGCAAAGCTGGTCC
TGAACACAGCTGTCCCAGATCGGCTGCCTGCCAGGGAAAGGGAGATGGAT
GTCATCAGGAATTTCTTGAGGGAACACATCTGTGGGAAAAAAGCTGGAAG
CCTTTACCTTTCTGGTGCTCCTGGAACTGGAAAAACTGCCTGCTTAAGCC
GGATTCTGCAAGACCTCAAGAAGGAACTGAAAGGCTTTAAAACTATCATG
CTGAATTGCATGTCCTTGAGGACTGCCCAGGCTGTATTCCCAGCTATTGC
TCAGGAGATTTGTCAGGAAGAGGTATCCAGGCCAGCTGGGAAGGACATGA
TGAGGAAATTGGAAAAACATATGACTGCAGAGAAGGGCCCCATGATTGTG
TTGGTATTGGACGAGATGGATCAACTGGACAGCAAAGGCCAGGATGTATT
GTACACGCTATTTGAATGGCCATGGCTAAGCAATTCTCACTTGGTGCTGA
TTGGTATTGCTAATACCCTGGATCTCACAGATAGAATTCTACCTAGGCTT
CAAGCTAGAGAAAAATGTAAGCCACAGCTGTTGAACTTCCCACCTTATAC
CAGAAATCAGATAGTCACTATTTTGCAAGATCGACTTAATCAGGTATCTA
GAGATCAGGTTCTGGACAATGCTGCAGTTCAATTCTGTGCCCGCAAAGTC
TCTGCTGTTTCAGGAGATGTTCGCAAAGCACTGGATGTTTGCAGGAGAGC
TATTGAAATTGTAGAGTCAGATGTCAAAAGCCAGACTATTCTCAAACCAC
TGTCTGAATGTAAATCACCTTCTGAGCCTCTGATTCCCAAGAGGGTTGGT
CTTATTCACATATCCCAAGTCATCTCAGAAGTTGATGGTAACAGGATGAC
CTTGAGCCAAGAAGGAGCACAAGATTCCTTCCCTCTTCAGCAGAAGATCT
TGGTTTGCTCTTTGATGCTCTTGATCAGGCAGTTGAAAATCAAAGAGGTC
ACTCTGGGGAAGTTATATGAAGCCTACAGTAAAGTCTGTCGCAAACAGCA
GGTGGCGGCTGTGGACCAGTCAGAGTGTTTGTCACTTTCAGGGCTCTTGG
AAGCCAGGGGCATTTTAGGATTAAAGAGAAACAAGGAAACCCGTTTGACA
AAGGTGTTTTTCAAGATTGAAGAGAAAGAAATAGAACATGCTCTGAAAGA
TAAAGCTTTAATTGGAAATATCTTAGCTACTGGATTGCCTTAAATTCTTC
TCTTACACCCCACCCGAAAGTATTCAGCTGGCATTTAGAGAGCTACAGTC
TTCATTTTAGTGCTTTACACATTCGGGCCTGAAAACAAATATGACCTTTT
TTACTTGAAGCCAATGAATTTTAATCTATAGAITCTTTAATATTAGCACA
GAATAATATCTTTGGGTCTTACTATTTTTACCCATAAAAGTGACCAGGTA
GACCCTTTTTAATTACATTCACTACTTCTACCACTTGTGTATCTCTAGCC
AATGTGCTTGCAAGTGTACAGATCTGTGTAGAGGAATGTGTGTATATTTA
CCTCTTCGTTTGCTCAAACATGAGTGGGTATTTTTTTGTTTGTTTTTTTT
TGTTGTTGTTGTTTTTGAGGCGCGTCTCACCCTGTTGCCCAGGCTGGAGT
GCAATGGCGCGTTCTCTGCTCACTACAGCACCCGCTTCCCAGGTTGAAGT
GATTCTCTTGCCTCAGCCTCCCGAGTAGCTGGGATTACAGGTGCCCACCA
CCGCGCCCAGCTAATTTTTTAATTTTTAGTAGAGACAGGGTTTTACCATG
TTGGCCAGGCTGGTCTTGAACTCCTGACCCTCAAGTGATCTGCCCACCTT
GGCCTCCCTAAGTGCTGGGATTATAGGCGTGAGCCACCATGCTCAGCCAT
TAAGGTATTTTGTTAAGAACTTTAAGTTTAGGGTAAGAAGAATGAAAATG
ATCCAGAAAAATGCAAGCAAGTCCACATGGAGATTTGGAGGACACTGGTT
AAAGAATTTATTTCTTTGTATAGTATACTATGTTCATGGTGCAGATACTA
CAACATTGTGGCATTTTAGACTCGTTGAGTTTCTTGGGCACTCCCAAGGG
CGTTGGGGTCATAAGGAGACTATAACTCTACAGATTGTGAATATATTTAT
TTTCAAGTTGCATTCTTTGTCTTTTTAAGCAATCAGATTTCAAGAGAGCT
CAAGCTTTCAGAAGTCAATGTGAAAATTCCTTCCTAGGCTGTCCCACAGT
CTTTGCTGCCCTTAGATGAAGCCACTTGTTTCAAGATGACTACTTTGGGG
TTGGGTTTTCATCTAAACACATTTTTCCAGTCTTATTAGATAAATTAGTC
CATATGGTTGGTTAATCAAGAGCCTTCTGGGTTTGGTTTGGTGGCATTAA
ATGG

SEQ ID. NO. 6

>gi|4502703|ref|NP_001245.1| CDC6 homolog
[Homo sapiens]
MPQTRSQAQATISFPKRKLSRALNKAKNSSDAKLEPTNVQTVTCSPRVKA
LPLSPRKRLGDDNLCNTPHLPPCSPPKQGKKENGPPHSHTLKGRRLVFDN
QLTIKSPSKRELAKVHQNKILSSVRKSQEITTNSEQRCPLKKESACVRLF
KQEGTCYQQAKLVLNTAVPDRLPAREREMDVIRNFLREHICGKKAGSLYL
SGAPGTGKTACLSRILQDLKKELKGFKTIMLNCMSLKTAQAVFPAIAQEI
CQEEVSRPAGKDMMRKLEKHMTAEKGPMIVLVLDEMDQLDSKGQDVLYTL
FEWPWLSNSHLVLIGIANTLDLTDRILPRLQAREKCKPQLLNFPPYTRNQ
IVTILQDRLNQVSRDQVLDNAAVQFCARKVSAVSGDVRKALDVCRRAIEI
VESDVKSQTILKPLSECKSPSEPLIPKRVGLIHISQVISEVDGNRMTLSQ
EGAQDSFPLQQKILVCSLMLLIRQLKIKEVTLGKLYEAYSKVCRKQQVAA
VDQSECLSLSGLLEARGILGLKRNKETRLTKVFFKIEEKEIEHALKDKAL
IGNILATGLP

SEQ ID NO. 7

>gi|4502858|ref|NM_001827.1| Homo sapiens CDC28
protein kinase regulatory subunit 2 (CKS2), mRNA
AGTCTCCGGCGAGTTGTTGCCTGGGCTGGACGTGGTTTTGTCTGCTGCGC
CCGCTCTTCGCGCTCTCGTTTCATTTTCTGCAGCGCGCCACGAGGATGGC
CCACAAGCAGATCTACTACTCGGACAAGTACTTCGACGAACACTACGAGT
ACCGGCATGTTATGTTACCCAGAGAACTTTCCAAACAAGTACCTAAAACT
CATCTGATGTCTGAAGAGGAGTGGAGGAGACTTGGTGTCCAACAGAGTCT
AGGCTGGGTTCATTACATGATTCATGAGCCAGAACCACATATTCTTCTCT
TTAGACGACCTCTTCCAAAAGATCAACAAAAATGAAGTTTATCTGGGGAT
CGTCAAATCTTTTTCAAATTTAATGTATATGTGTATATAAGGTAGTATTC
AGTGAATACTTGAGAAATGTACAAATCTTTCATCCATACCTGTGCATGAG
CTGTATTCTTCACAGCAACAGAGCTCAGTTAAATGCAACTGCAAGTAGGT
TACTGTAAGATGTTTAAGATAAAAGTTCTTCCAGTCAGTTTTTCTCTTAA
GTGCCTGTTTGAGTTTACTGAAACAGTTTACTTTTGTTCAATAAAGTTTG
TATGTTGCATTTAAAAAAAAAAAAAAA

SEQ ID NO. 8

>gi|4502859|ref|NP_001818.1| CDC28 protein
kinase 2 [Homo sapiens]
MAHKQIYYSDKYFDEHYEYRHVMLPRELSKQVPKTHLMSEEEWRRLGVQQ
SLGWVHYMIHEPEPHILLFRRPLPKDQQK

SEQ ID NO. 9

>gi|51468493|ref|XM_166103.5| PREDICTED: Homo
sapiens DNA2 DNA replication helicase 2-like
(yeast) (DNA2L), mRNA
ATGAAGACTCCCTGTATTCCCAGTCCTAAGCAAGGGAGCAAAGGCAGGGG
ACAGAGCCCTGCTGCTCAGGTGAGGGGCCCCACGTGGAACGCGCCGGCGC
GGGAGGGGCGGCCTGGCGCAGGTCATTTGGGACATCTGCGGGTTGGCGCA
TGCGCGCGAGGTGCGCAGGCTCGCGCCTTTTTCCCTTTTCTAAGCTTTCT
GTGTTACCCCCGGTTCCGCTGTCTTTTCTGTCTACAGTTTGCGATCCCCG
CGTCCAGGATGGAGCAGCTGAACGAACTGGAGCTGCTGATGGAGAAGAGT
TTTTGGGAGGAGGCGGAGCTGCCGGCGGAGCTATTTCAGAAGAAAGTGGT
AGCTTCCTTTCCAAGAACAGTTCTGAGCACAGGAATGGATAACCGGTACC
TGGTGTTGGCAGTCAATACTGTACAGAACAAAGAGGGAAACTGTGAAAAG
CGCCTGGTCATCACTGCTTCACAGTCACTAGAAAATAAAGAACTATGCAT
CCTTAGGAATGACTGGTGTTCTGTTCCAGTAGAGCCAGGAGATATCATTC
ATTTGGAGGGAGACTGCACATCTGACACTTGGATAATAGATAAAGATTTT
GGATATTTGATTCTGTATCCAGACATGCTGATTTCTGGCACCAGCATAGC
CAGTAGTATTCGATGTATGAGAAGAGCTGTCCTGAGTGAAACTTTTAGGA
GCTCTGATCCAGCCACACGCCAAATGCTAATTGGTACGGTTCTCCATGAG
GTGTTTCAAAAAGCCATAAATAATAGCTTTGCCCCAGAAAAGCTACAAGA
ACTTGCTTTTCAAACAATTCAAGAAATAAGACATTTGAAGGAAATGTACC
GCTTAAATCTAAGTCAAGATGAAATAAAACAAGAAGTAGAGGACTATCTT
CCTTCGTTTTGTAAATGGGCAGGAGATTTCATGCATAAAAACACTTCGAC
TGACTTCCCTCAGATGCAGCTCTCTCTGCCAAGTGATAATAGTAAGGATA
ATTCAACATGTAACATTGAAGTCGTGAAACCAATGGATATTGAAGAAAGC
ATTTGGTCCCCTAGGTTTGGATTGAAAGGCAAAATAGATGTTACAGTTGG
TGTGAAAATACATCGAGGGTATAAAACAAAATACAAGATAATGCCGCTGG
AACTTAAAACTGGCAAAGAATCAAATTCTATTGAACACCGTAGTCAGGTT
GTTCTGTACACTCTACTAAGCCAAGAGAGAAGAGCTGATCCAGAGGCTGG
CTTGCTTCTCTACCTCAAGACTGGTCAGATGTACCCTGTGCCTGCCAACC
ATCTAGATAAAAGAGAATTATTAAAGCTAAGAAACCAGATGGCATTCTCA
TTGTTTCACCGTATTAGCAAATCTGCTACTAGACAGAAGACACAGCTTGC
TTCTTTGCCACAAATAATTGAGGAAGAGAAAACTTGTAAATATTGTTCAC
AAATTGGCAATTGTGCTCTTTATAGCAGAGCAGTTGAACAACAGATGGAT
TGTAGTTCAGTCCCAATTGTGATGCTGCCCAAAATAGAAGAAGAAACCCA
GCATCTGAAGCAAACACACTTAGAATATTTCAGCCTTTGGTGTCTAATGT
TAACCCTGGAGTCACAATCGAAGGATAATAAAAAGAATCACCAAAATATC
TGGCTAATGCCTGCTTCGGAAATGGAGAAGAGTGGCAGTTGCATTGGAAA
CCTGATTAGAATGGAACATGTAAAGATAGTTTGTGATGGGCAATATTTAC
ATAATTTCCAATGTAAACATGGTGCCATACCTGTCACAAATCTAATGGCA
GGTGACAGAGTTATTGTAAGTGGAGAAGAAAGGTCACTGTTTGCTTTGTC
TAGAGGATATGTGAAGGAGATTAACATGACAACAGTAACTTGTTTATTAG
ACAGAAACTTGTCGGTCCTTCCAGAATCAACTTTGTTCAGATTAGACCAA
GAAGAAAAAAATTGTGATATAGATACCCCATTAGGAAATCTTTCCAAATT
GATGGAAAACACGTTTGTCAGCAAAAAACTTCGAGATTTAATTATTGACT
TTCGTGAACCTCAGTTTATATCCTACCTTAGTTCTGTTCTTCCACATGAT
GCAAAGGATACAGTTGCCTGCATTCTAAAGGGTTTGAATAAGCCTCAGAG
GCAAGCGATGAAAAAGGTACTTCTTTCAAAAGACTACACACTCATCGTGG
GTATGCCTGGGACAGGAAAAACAACTACGATATGTACTCTCGTAAGAATT
CTCTACGCCTGTGGTTTTAGCGTTTTGTTGACCAGCTATACACACTCTGC
TGTTGACAATATTCTTTTGAAGTTAGCCAAGTTTAAAATAGGATTTTTGC
GTTTGGGTCAGATTCAGAAGGTTCATCCAGCTATCCAGCAATTTACAGAG
CAAGAAATTTGCAGATCAAAGTCCATTAAATCCTTAGCTCTTCTAGAAGA
ACTCTACAATAGTCAACTTATAGTTGCAACAACATGTATGGGAATAAACC
ATCCAATATTTTCCCGTAAAATTTTTGATTTTTGTATTGTGGATGAAGCC
TCTCAAATTAGCCAACCAATTTGTCTGGGCCCCCTTTTTTTTTCACGGAG
ATTTGTGTTAGTGGGGGACCATCAGCAGCTTCCTCCCCTGGTGCTAAACC
GTGAAGCAAGAGCTCTTGGCATGAGTGAAAGCTTATTCAAGAGGCTGGAG
CAGAATAAGAGTGCTGTTGTACAGTTAACCGTGCAGTACAGAATGAACAG
TAAAATTATGTCCTTAAGTAATAAGCTGACCTATGAGGGCAAGCTGGAGT
GTGGATCAGACAAAGTGGCCAATGCAGTGATAAACCTACGTCACTTTAAA
GATGTGAAGCTGGAACTGGAATTTTATGCTGACTATTCTGATAATCCTTG
GTTGATGGGAGTATTTGAACCCAACAATCCTGTTTGTTTCCTTAATACAG
ACAAGGTTCCAGCGCCAGAACAAGTTGAAAAAGGTGGTGTGAGCAATGTA
ACAGAAGCCAAACTCATAGTTTTCCTAACCTCCATTTTTGTTAAGGCTGG
ATGCAGTCCCTCTGATATTGGTATTATTGCACCGTACAGGCAGCAATTAA
AGATCATCAATGATTTATTGGCACGTTCTATTGGGATGGTCGAAGTTAAT
ACAGTAGACAAATACCAAGGAAGGGACAAAAGTATTGTCCTAGTATCTTT
TGTTAGAAGTAATAAGGATGGAACTGTTGGTGAACTCTTGAAAGATTGGC
GACGTCTTAATGTTGCTATAACCAGAGCCAAACATAAACTGATTCTTCTG
GGGTGTGTGCCCTCACTAAATTGCTATCCTCCTTTGGAGAAGCTGCTTAA
TCATTTAAACTCAGAAAAATTAATCATTGATCTTCCATCAAGAGAACATG
AAAGTCTTTGCCACATATTGGGTGACTTTCAAAGAGAATAAAACACTATT
TCCCTTGCCTTTTCATACTAGGGCAGTATCTCCTCTAGCTAGTGCCCATA
CAGAAAATTCTATCACCATACAAAATTTAATGCAGTATTTATGTTTTAAA
GCACAGGTGTACCGAAAACTGTGAAAAGTCTGAATTTATGGGTTCTATGC
ATGCATTTTTGCCTAACCTAGAGAAAGAGTTTGATAAATTTTTACCAGCT
TTGAAGATGGATTAACTTTTGACTTTGAGCTTTAAACTTTTAAGTCAGAC
ATTTCAGGACTAATTTGATTTTGTAGATATCATTGTAAGAACTTTATTTG
AAAGACTGAATAAAGGGATTTGATTTGTTTTCATCATTTAAGCACAGTCT
TGTGATGATGAGAACATAAGTGTGATTCTTTTCTGTATTTTGAGGTCCCT
AATCCAAAGCCCATTTTGCTAGGATTTTTTCTGCTATCAGATGTGTTTTC
ACTCTAAACCTAGTCTTTTATGACATGAATTGATTACTTCCTGTTAATTT
TCTATCCTCCCTTACTATCCTCCTTTTTTGTTTTCAGTATTCAGTATTTC
AGTATTCTAGAGTAGATTTTGATATAAAAGAAAATAATTCTTACATCATC
TTTTGCAACAAATTTTGTTTTCTGAATTGATAATAAATTTAAAAAGTTGA
TTCCTATTTTCACATATGTTCATATGCCCCTATGTTTGGGGGTATCACTC
AGTTTTCCCTTTTTTGTGTAAAGATGTTTTGTAAAACAAAATTGTCTCAA
AGTGATTATATTATATATATAAAAAGTAACAGATTTTAACAAAGGTTAAA
AGATTCTTGGGGTAACAGATTCTTCTGGGGCTTGGAAATCTTCCATTTCT
CTTGAGGGTTTTTTTTAATGAGTGTTAAATATGTTAAAATTTTTATTTCT
ACCTCATGTGTTTTTTTAAATTATTACTTGAAGTTTTTTATTTAATAAAT
TTTTTCTACTAATGG

SEQ ID NO. 10

>gi|51468494|ref|XP_166103.4| PREDICTED: DNA2 DNA
replication helicase 2-like [Homo sapiens]
MKTPCIPSPKQGSKGRGQSPAAQVRGPTWNAPAREGRPGAGHLGHLRVGA
CARGAQARAFFPFLSFLCYPRFRCLFCLQFAIPASRMEQLNELELLMEKS
FWEEAELPAELFQKKVVASFPRTVLSTGMDNRYLVLAVNTVQNKEGNCEK
RLVITASQSLENKELCILRNDWCSVPVEPGDIIHLEGDCTSDTWIIDKDF
GYLILYPDMLISGTSIASSIRCMRRAVLSETFRSSDPATRQMLIGTVLHE
VFQKAINNSFAPEKLQELAFQTIQEIRHLKEMYRLNLSQDEIKQEVEDYL
PSFCKWAGDFMHKNTSTDFPQMQLSLPSDNSKDNSTCNIEVVKPMDIEES
IWSPRFGLKGkIDVTVGVKIHRGYKTKYKIMPLELKTGKESNSIEHRSQV
VLYTLLSQERRADPEAGLLLYLKTGQMYPVPANHLDKRELLKLRNQMAFS
LFHRISKSATRQKTQLASLPQIIEEEKTCKYCSQIGNCALYSRAVEQQMD
CSSVPIVMLPKIEEETQHLKQTHLEYFSLWCLMLTLESQSKDNKKNHQNI
WLMPASEMEKSGSCIGNLIRMEHVKIVCDGQYLHNFQCKHGAIPVTNLMA
GDRVIVSGEERSLFALSRGYVKEINMTTVTCLLDRNLSVLPESTLFRLDQ
EEKNCDIDTPLGNLSKLMENTFVSKKLRDLIIDFREPQFISYLSSVLPHD
AKDTVACILKGLNKPQRQAMKKVLLSKDYTLIVGMPGTGKTTTICTLVRI
LYACGFSVLLTSYTHSAVDNILLKLAKFKIGFLRLGQIQKVHPAIQQFTE
QEICRSKSIKSLALLEELYNSQLIVATTCMGINHPIFSRKIFDFCIVDEA
SQISQPICLGPLFFSRRFVLVGDHQQLPPLVLNREARALGMSESLFKRLE
QNKSAVVQLTVQYRMNSKIMSLSNKLTYEGKLECGSDKVANAVINLRHFK
DVKLELEFYADYSDNPWLMGVFEPNNPVCFLNTDKVPAPEQVEKGGVSNV
TEAKLIVFLTSIFVKAGCSPSDIGIIAPYRQQLKIINDLLARSIGMVEVN
TVDKYQGRDKSIVLVSFVRSNKDGTVGELLKDWRRLNVAITRAKHKLILL
GCVPSLNCYPPLEKLLNHLNSEKLIIDLPSREHESLCHILGDFQRE

In one embodiment, the level of other markers other than CDC2, CDC6, DNA2L or CKS2 may also be determined. For example, the level of ER-β may also be determined.

A “marker nucleic acid” includes a nucleic acid (e.g., mRNA, cDNA) encoded by or corresponding to a marker of the invention. Such marker nucleic acids include DNA (e.g., genomic DNA or cDNA) comprising the entire or a partial sequence of the marker or the complement of such a sequence. The marker nucleic acids also include RNA comprising the entire or a partial sequence of the marker or the complement of such a sequence.

A “marker protein” includes a protein encoded by or corresponding to a marker of the invention. A marker protein may comprise the entire or a partial sequence of the marker or a variant of the marker. For the avoidance of doubt, the terms “protein” and “polypeptide” are used interchangeably herein.

Thus, by determining the level of a particular marker, the level of one or more of the following moieties may, for example, be determined: full-length cDNA markers, full-length genomic DNA markers, fragments of the full-length cDNA markers or genomic DNA markers, and transcribed polynucleotides.

The level of a marker may be determined by any means known in the art. The level may be determined by, for example, determining the level of nucleic acid transcribed from a marker gene. Alternatively, or additionally, the level of specific proteins translated from mRNA transcribed from a marker genie may be determined. In yet another embodiment, the level of a metabolite which is produced directly (i.e., catalyzed) or indirectly or “consumed” by the corresponding marker protein could be determined.

An exemplary assay for determining the level of a marker involves obtaining a sample (e.g., a breast associated body fluid) of an individual and contacting the sample with a probe (e.g. antibody, oligonucleotide) capable of detecting the marker protein or marker nucleic acid (e.g., mRNA, genomic DNA, or cDNA) under appropriate conditions and for a time sufficient allow the marker and probe to interact and bind, thus forming a complex that can be removed and/or detected in the reaction mixture. The detection methods of the invention can thus be used to detect mRNA, protein, cDNA, or genomic DNA, for example, in a biological sample in vitro as well as in vivo.

These assays can be conducted in a variety of way. For example, one method to conduct such an assay would involve anchoring the marker or probe onto a solid support and detecting target marker/probe complexes anchored on the solid phase at the end of the reaction. In one embodiment of such a method, a sample of an individual, which is to be assayed for presence, amount and/or concentration of marker, can be anchored onto a solid support. In another embodiment, the reverse situation is possible, in which the probe can be anchored to a solid support (e.g. a nylon membrane or a chip) and a sample of an individual can be allowed to react as an unanchored component of the assay. In one embodiment, the probes may be immobilized on a microarray.

In order to conduct assays with the above-mentioned approaches, the non-immobilized component is added to the solid support upon which the second component is anchored. After the reaction is complete, uncomplexed components may be removed (e.g., by washing) under conditions such that any complexes formed will remain immobilized upon the solid support. The detection of marker/probe complexes anchored to the solid support can be accomplished in a number of methods.

In a preferred embodiment, the probe, when it is the unanchored assay component, can be labeled for the purpose of detection and readout of the assay, either directly or indirectly, with a detectable label. It is also possible to directly detect marker/probe complex formation without further manipulation or labeling of either component (marker or probe), for example by utilizing the technique of fluorescence energy transfer (see; for example, Lakowicz et al., U.S. Pat. No. 5,631,169; Stavrianopoulos, et al., U.S. Pat. No. 4,868,103).

The level of expression of specific marker genes can, for example, be accomplished by determining the amount of mRNA (or polynucleotides derived therefrom) present in a sample. A skilled artisan can readily adapt known mRNA detection methods for use in detecting the level of mRNA encoded by a marker of the present invention.

Many techniques for the detection and quantification of mRNA levels involve contacting the mRNA with a nucleic acid molecule probe) that can hybridize to the mRNA (or polynucleotide derived therefrom). The nucleic acid probe can be, for example, a polynucleotide of at least 7, 10, 15, 17, 18, 20, 25, 30, 40, 50, 100, 500, nucleotide residues in length. Probes may include, but are not limited to, oligonucleotides, cDNA, or RNA. Probes may contain a detectable label, such as a fluorescent or chemiluminescent label. When a method of assessing marker expression is used which involves hybridization of one nucleic acid with another, it is preferred that the hybridization be performed under stringent hybridization conditions.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor; N.Y, 1989); Berger and Kimmel Methods in Enzymology, Vol, 152, Guide to Molecular Cloning Techniques (Academic Press, Inc., San Diego, Calif., 1987); Young and Davis, P.N.A.S, 80: 1194 (1983). Methods and apparatuss for carrying out repeated and controlled hybridization reactions have been described in U.S. Pat. Nos. 5,871,928, 5,874,219, 6,045,996 and 6,386,749, 6,391,613 each of which are incorporated herein by reference.

The present invention contemplates signal detection of hybridization between ligands in certain preferred embodiments. See U.S. Pat. Nos. 5,143,854; 5,578,832; 5,631,734; 5,834,758; 5,936,324; 5,981,956; 6,025,601; 6,141,096; 6,185,030; 6,201,639; 6,218,803; and 6,215,625, U.S. Patent Application No. 60/364,731 and PCT Application No. PCT/US99/06097 (published as WO99/47964), each of which also is hereby incorporated by reference in its entirety for all purposes.

Methods and apparatus for signal detection and processing of intensity data are disclosed in for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent Application 60/364,731 and in PCT Application PCT/US99/06097 (published as WO99/47964), each of which also is hereby incorporated by reference in its entirety for all purposes.

Any method for determining nucleic acid or protein levels can be used in the present invention and the examples described herein are not intended to be limiting.

In one format, mRNA is immobilized on a solid support and contacted with a probe, for example by running the isolated mRNA on an agarose gel and transferring the mRNA from the gel to a solid support such as a filter. Nucleic acid probes representing one or more markers are then hybridized to the filter by northern hybridization, and the amount of marker-derived RNA is determined. Such determination can be visual, or machine-aided, for example, by use of a densitometer. In an alternative format, the probe(s) are immobilized on a solid support and the nucleic acid is contacted with the probe(s), for example, in an Affymetrix gene chip array. The present invention also contemplates sample preparation methods in certain preferred embodiments. For example, prior to or concurrent with gene expression analysis, the sample may be amplified by a variety of mechanisms, some of which may employ amplification techniques such as PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, CA, 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202; 4,683,195, 4,800,159 4,965,183, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et at. Gene 89:117 (1990)), transcription-amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. No. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

In one embodiment of the present invention, the level of a marker protein is determined. A preferred agent for determining the level of a marker protein of the invention is an antibody capable of binding to such a protein or a fragment thereof, preferably an antibody with a detectable label.

Suitable antibodies can be produced using techniques well known to those of skill in the art and disclosed in, for example, U.S. Pat. Nos. 4,011,308; 4,722,890; 4,016,043; 3,876,504; 3,770,380; and 4,372,745. Preferably, monoclonal antibodies are employed. Monoclonal antibodies are generally prepared using the method of Kohler & Milstein (1975) Nature 256:495-497, or a modification thereof. Typically, a mouse or rat is immunized with the protein of interest. Rabbits may also be used. The spleen (and optionally several large lymph nodes) is removed and dissociated into single cells. If desired, the spleen cells may be screened (after removal of non-specifically adherent cells) by applying a cell suspension to a plate or well coated with the antigen. B-cells, expressing membrane-bound immunoglobulin specific for the antigen, will, bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution; and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice).

A variety of formats can be employed, to determine whether a sample contains a protein that binds to a given antibody. Examples of such formats include, but are not limited to, enzyme immunoassay (EIA), radioimmunoassay (RIA), enzyme linked immunosorbent assays (ELISAs), Western blots, immunoprecipitation and immunofluorescence. In such uses, it is generally preferable to immobilize either the antibody or proteins on a solid support. Suitable supports include any support capable of binding an antigen or an, antibody. In one embodiment, marker-derived protein levels can be determined by constructing an antibody microarray in which binding sites comprise immobilized, preferably monoclonal, antibodies specific to a marker protein. By utilising antibodies which are specific for different marker proteins, the level of more than one marker protein may be determined using a single microarray.

In addition, preferred in vivo techniques for detection of a marker protein include introducing into a subject a labeled antibody directed against a marker protein. For example, the antibody can be labeled with a radioactive marker whose presence and location in a subject can be detected by standard imaging techniques.

The present invention can employ solid supports, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques may be applied to polypeptide (e.g. antibody) arrays.

In preferred embodiments, polynucleotide microarrays are used to determine the level of a marker. In this way, the expression status of more than one marker may be assessed simultaneously. In a specific embodiment, the invention provides for arrays comprising probes hybridizable to at least one, two or three of (and preferably to all four of) CDC2, CDC6, CKS2 and DNA2L. The microarrays may comprise further probes suitable for detecting one or more markers additional to CDC2, CDC6, CKS2 or DNA2L, e.g. for detecting ER-β.

Microarrays may be prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (eg., by PCR), or nonenzymatically in vitro.

The probe or probes used in the methods of the invention are preferably immobilized to a solid support which may be either porous or non-porous. For example, the probes of the invention may be polynucleotide sequences which are attached to a nitrocellulose or nylon membrane or filter covalently at either the 3′ or the 5′ end of the polynucleotide. Alternatively, the solid support may be a glass or plastic surface. In a particularly preferred embodiment, hybridization levels are measured to microarrays of probes consisting of a solid support on the surface of which are immobilized a population of polynucleotides, such as a population of DNA or DNA mimics, or, alternatively, a population of RNA or RNA mimics. The solid support may be a nonporous or, optionally, a porous material such as a gel.

A skilled artisan will also appreciate that it may be desirable to include positive control probes, e.g., probes known to be complementary and hybridizable to sequences in the target polynucleotide molecules, and/or negative control probes, e.g., probes known to not be complementary and hybridizable to sequences in the target polynucleotide molecules, on the array.

The present invention may also make use of the several embodiments of the array or arrays and the processing described in U.S. Pat. Nos. 5,545,531 and 5,874,219. These patents are incorporated herein by reference in their entireties for all purposes.

The present invention may make use of various program products and software for a variety of purposes, such as probe design, management of data, analysis, and instrument operation. See, U.S. Pat. Nos. 5,795,716; 5,733,729, 5,974,164, 6,066,454, 6,090,555, 6,185,561, 6,188,783, 6,223,127, 6,229,911 and 6,308,170.

In some embodiments, the expression levels of marker nucleic acids may be used to generate expression profiles. An expression profile of a particular sample is essentially a “fingerprint” of the state of the sample—while two states may have any particular gene similarly expressed, the evaluation of a number of genes simultaneously allows the generation of a gene expression profile that is characteristic of the state of the cell. This allows normal tissue to be distinguished from, for example, cancerous tissue, or cancer tissue to be compared with tissue from surviving cancer patients. Comparing expression profiles in different cancer states identifies genes (e.g. up- and down-regulated genes) that are important in each of these states. Molecular profiling may distinguish subtypes of a currently collective disease designation, e.g., different forms or stages of a cancer. Metastatic tissue may also be analyzed to determine the stage of cancer in the tissue, or origin of primary tumor, e.g., metastasis from a remote primary site.

By assessing the level of one or more of the markers of the invention (i.e. CDC2, CDC6, DNA2L and CKS2) information may be gleaned concerning ER-β function. As mentioned above, the level of the markers of the invention have been correlated with clinical outcome and as discussed in the Examples section below, the level of the markers have been found to correlate with the following: (i) tumor grade (grade 1, 2, or 3); (ii) disease recurrence within 10 years of surgery; (iii) death within 10 years of surgery; and (iv) and p53 mutation status.

Since the levels of CDC2, CDC6, DNA2L and CKS2 are inversely correlated with ER-β function then a low level of one or more of these markers is indicative of a good prognosis. A low level of one or more of these markers is also indicative of the patient having a good response to adjuvant endocrine therapy and thus measurement of one or more of the markers of the invention may be used in treatment selection.

In addition to determining the level of a marker of the invention, other markers (e.g. a marker as included in Table 3) and other factors may also be taken into account e.g. gender, age, previous cancer history, benign breast disease, hereditary factors (family history of cancer), early age at menarche (first menstrual period), late age at menopause, late age at first full-term pregnancy, obesity, low physical activity, use of postmenopausal hormone replacement therapy, use of oral contraceptives, exposure to ionizing radiation, dietary practices, or alcohol consumption.

The methods, arrays, kits etc. of the invention may be used to evaluate a patient before, during and after treatment, for example, to evaluate the reduction in tumor burden. The methods of the invention may be employed in vivo or in vitro. The methods of the invention may employ a kit or array as described herein.

It is envisaged that the invention will find utility in at least the following areas:
Prognosis
Characterization of the cancer (e.g. stage, grade, or ER-β level)
Treatment selection
Assessment of treatment efficacy
Assessment of cancer progression of regression
Identification and development of new or existing treatments for cancer
Assessing the carcinogenic potential of a test compound

The invention is envisaged as finding particular utility with respect to breast cancer but may also find utility with respect to other cancers. Thus, the cancer may for example be adenocarcinoma, leukemia, lymphoma, melanoma, myeloma, sarcoma or cancers of the blood, bone, bone marrow, brain, breast, gastrointestinal tract (esophagus, stomach, small intestine or colon), heart, kidney, liver, lung, lymph, muscle, nerve, ovary, pancreas, prostate, skin, spleen, testis, or uterus. In one embodiment, the cancer is an endocrine-related cancer. In one embodiment, the cancer is a primary cancer. In one embodiment the cancer is breast cancer. “Breast cancer” as used herein includes carcinomas, (e.g., carcinoma in situ, invasive carcinoma, metastatic carcinoma) and pre-malignant conditions. In one embodiment, the cancer is a primary breast cancer. In one embodiment the cancer is ER-α positive. The terms tumor and cancer and used interchangeably herein unless the context indicates otherwise.

It is envisaged that the present invention will find particular utility in relation to ER-α positive tumors and in the area of adjuvant endocrine hormone therapy. In particular, it is envisaged that the invention will find particular utility in respect of patients selected for adjuvant endocrine hormone therapy (e.g. patients which are receiving or which have previously received adjuvant endocrine hormone therapy or which have been identified as patients for which adjuvant endocrine hormone therapy would be appropriate for).

In one embodiment of the invention, the level of one or more of the markers of the invention is used in predicting the prognosis of an individual. The level of one or more of the markers may provide information on, inter alia, patient survival, likelihood of disease recurrence and disease metastasis (patient survival, disease recurrence and disease metastasis may be assessed in relation to a defined timepoint, e.g. at a given number of years after cancer surgery (e.g. surgery to remove one or more tumors) or after initial diagnosis). In one embodiment, the prognosis comprises a prediction of DSS or DFS.

Depending on the level of the one or markers of the invention in the patient sample, individual appropriate treatment may be selected for the patient. For instance, the continuation of existing treatment may be deemed appropriate and/or the commencement of additional or a different treatment for that individual may be deemed appropriate.

The markers of the invention may be useful as pharmacogenomic markers. As used herein, a “pharmacogenomic marker” is an objective biochemical marker whose expression level correlates with a specific clinical drug response or other treatment response or susceptibility in an individual. The presence or quantity of the pharmacogenomic marker expression is related to the predicted response of the patient and more particularly the patient's tumor to treatment, e.g. with a specific drug or class of drugs, e.g. adjuvant endocrine therapy. By assessing the presence or quantity of the expression of one or more pharmacogenomic markers in a patient, a treatment which is appropriate for the patient may be selected.

For example, based on the level of marker of the invention, a drug or course of treatment may be selected that is optimized for the treatment of the specific tumor likely to be present in the patient. The use of a pharmacogenomic marker therefore permits selecting or designing the most appropriate treatment for each cancer patient without trying different drugs or treatment regimes.

Knowledge of the level of a marker of the invention (optionally in conjunction with other factors e.g. cell morphology) may be used to aid in the characterization of a cell/tumor, for instance its ER-β level and/or the grade of the tumor (e.g. grade 1, 2, 3 according to the Scarff-Bloom-Richardson grade system). Information regarding the characterization of a cell/tumor may for example be used in the prognosis of the patient and/or the selection of appropriate treatment for the patient.

Doctors use tumor grade and many other factors, such as cancer stage, to develop an individual treatment plan for the patient and to predict the patient's prognosis. Generally, a lower grade indicates a better prognosis (the likely outcome or course of a disease; the chance of recovery or recurrence).

In one embodiment, the level of one or more markers of the invention is used in the characterization of a tumor according to the Elston-Ellis modification of the Scarff-Bloom-Richardson grade system. Briefly, according to the Scarff-Bloom-Richardson grade system there are the following grades:
Grade 1 (lowest): Well-differentiated breast cells; cells generally appear normal and are not growing rapidly; cancer arranged in small tubules. 5 years survival: 95%; 7 year survival: 90%;
Grade 2 Moderately differentiated breast cells; have characteristics between Grade 1 and Grade 3 tumors. 5 years survival 75%; 7 year survival: 63%;
Grade 3 (highest). Poorly-differentiated breast cells; cells do not appear normal and tend to grow and spread more aggressively; 5 years survival: 50%; 7 year survival 45%.

The Elston-Ellis modification provides a somewhat more objective criteria for the three component elements of grading and specifically addresses mitosis, counting in a more rigorous fashion. For example hyperchromatic nuclei and apoptotic cells which are counted in the original SBR system stem are excluded in the Elston-Ellis modification and the area being assessed is specifically defined in square millimeters. These modifications have enhanced reproducibility of grading among pathologists and to a considerable extent have fostered acceptance of grading by clinicians (Elston, C. W. and Ellis I. O. (1991) Pathological prognostic factors in breast cancer. I. The value of histological grade in breast cancer: experience from a large study with long-term follow-up. Histopathology, 19, 403-10.

In one embodiment of the invention, the level, of one or more markers of the invention is used in the staging of a tumour. Staging describes the extent or severity of an individual's cancer based on the extent of the original (primary) tumor and the extent of spread in the body. Staging is important as it helps the doctor plan a person's treatment and the stage can be used to estimate the person's prognosis (likely outcome or course of the disease).

The common elements considered in most staging systems are: location of the primary tumor; tumor size and number of tumors, lymph node involvement (spread of cancer into lymph nodes), cell type and tumor grade and presence or absence of metastasis.

The TNM system is one of the most commonly used, staging systems. The TNM system is based on the extent of the tumor (T), the extent of spread to the lymph nodes (N), and the presence of metastasis (M). A number is added to each letter to indicate the size or extent of the tumor and the extent of spread.

In one embodiment of the invention, the knowledge of the level of a marker of the invention may be used to decide whether a patient should be treated with adjuvant endocrine therapy (e.g. tamoxifen).

The level of a maker of the invention may be measured at one or more time points. By monitoring the level of one or more of the markers of the invention, cancer progression (cancer progression as used herein includes cancer growth and metastasis), cancer regression and treatment efficacy may be assessed. The progression or regression of a cancer or the efficacy of a treatment may for example be assessed by using the grading or staging system as discussed above.

In one embodiment of the invention, the level of a marker of the invention may be used to help identify or develop a new or existing treatment for cancer. For example, gene expression profiles can allow screening of drug candidates with the goal of mimicking or altering a particular expression profile. This may be done by designing a microarray (for example) comprising one or more probes capable of hybridizing with at least one marker nucleic acid of the invention. The microarray can then be used to screen drug candidates that alter the expression of CDC2, CDC6, DNA2L and/or CKS2 (and optionally also ER-β). Screening assays may also be carried out at the protein level, i.e. the effect of a candidate agent on the level of one or more marker proteins of the present invention may be determined.

In a preferred embodiment, gene expression profiles are used, preferably in conjunction with high throughput screening techniques, to allow monitoring for expression profile genes after treatment with a candidate agent, for example.

Assessing the influence of agents (e.g., drug compounds) on the level of expression of a marker of the invention can be applied not only in basic drug screening or assessing the efficacy of treatment in an individual, but also in clinical trials. For example, the effectiveness of an agent to affect marker expression can be monitored in clinical trials of subjects receiving cancer treatment. In one embodiment, the present invention provides, a method for assessing the effectiveness of treatment of a subject with a candidate agent (e.g., an agonist, antagonist, peptidomimetic, protein, peptide, nucleic acid, small molecule, or other drug candidate) comprising the steps of obtaining a pre-administration sample from a subject prior to administration of the agent; (ii) detecting the level of expression of one or more selected markers of the invention in the pre-administration sample; (iii) obtaining one or more post-administration samples from the subject; (iv) detecting the level of expression of the marker(s) in the post-administration samples; (v) comparing the level of expression of the marker(s) in the pre-administration sample with the level of expression of the marker(s) in the post-administration sample or samples; and optionally (vi) altering the administration of the agent to the subject accordingly. For example, increased expression of the marker(s) during the course of treatment may indicate ineffective dosage and the desirability of increasing the dosage. Conversely, decreased expression of the marker(s) may indicate efficacious treatment and no need to change dosage. In an alternative embodiment, the level of expression of the marker(s) in the post-administration samples (step iv) may be detected at multiple time points. The multiple time points may be evenly spaced or non-evenly spaced or they may be chosen randomly.

In one embodiment, the influence of a candidate agent or a known drug on marker expression may be determined by comparing the level of one or more markers of the invention in one or more patients who have received the candidate agent or known drug, with the level of the one or more markers in one or more patients who have not received the candidate agent or known drug.

The invention additionally provides a method for assessing progression or regression of cancer in a patient, the method comprising: determining the level of at least one marker of the invention in a sample from the patient; repeating the marker level assessment at a subsequent time point; and comparing the level of the marker(s) in the first and second steps, thereby assessing the progression or regression of cancer in the patient. A significantly higher level of expression of the marker in the sample at the subsequent time point from that of the sample at the first time point is an indication that the cancer has worsened in the patient, whereas a significantly lower level of expression is an indication that the breast cancer has regressed. In one embodiment, the patient has undergone surgery to remove a tumor between the first point in time and the subsequent point in time. In an alternative embodiment, the subsequent time point may comprise multiple time points, i.e. the detection of expression step may be carried out at multiple time points until such a point in time when the difference in the level of expression of the marker between the first time point and the subsequent time point is significant.

In one embodiment of the invention there is provided a method which comprises comparing expression of at least one marker of the invention in a first sample (e.g. breast cell sample) and maintained in the presence of a candidate agent and expression of the at least one marker in a second sample (e.g. breast cell sample) and maintained in the absence of the candidate agent. A significantly reduced expression of the marker(s) of the invention in the presence of the candidate agent is an indication that the candidate agent inhibits cancer. Cell samples may be used which may for example, be aliquots of single sample of normal cells obtained from a patient, pooled samples of normal cells obtained from a patient, cells of a normal cell line, aliquots of a single sample of cancer cells obtained from a patient, pooled samples of cancer cells obtained from a patient, cells of a cancer cell line, or the like. In one embodiment, the samples are cancer cells obtained from a patient and one or more of a plurality of agents known to be effective for inhibiting cancer are tested in order to identify the agent which is likely to best inhibit the cancer in the patient.

The level of the one or more markers of the invention may be compared with a control for the one or more of said markers.

In one embodiment, the marker level is compared to the level of the marker from a control, wherein the control comprises one or more tumor samples (e.g. breast cancer samples) taken from one or more patients determined as having a good prognosis (“good prognosis” control) or a poor prognosis (“poor prognosis” control), or both. Alternatively or additionally, the control may comprise one or more tumor samples (e.g. breast cancer samples) taken from one or more patients determined as being of a particular tumor type (e.g. grade, stage, p53 mutation status).

A good or bad prognosis may, for example, be assessed in terms of patient survival, likelihood of disease recurrence or disease metastasis (patient survival, disease recurrence and metastasis may for example be assessed in relation to a defined timepoint, e.g. at a given number of years after cancer surgery (e.g. surgery to remove one or more tumors) or after initial diagnosis). In one embodiment, a good or bad prognosis may be assessed in terms of DSS or DFS.

The control may comprise data obtained at the same time (e.g. in the same hybridization experiment) as the patient's individual data; or may be a stored value or set of values e.g. stored on a computer, or on computer-readable media. If the latter is used, new patient data for the selected marker(s), obtained from initial or follow-up samples, can be compared to the stored data for the same marker(s) without the need for additional control experiment.

In one aspect of the invention there is provided a kit. The kit comprises a reagent for use in determining the level of at least one marker of the invention. The kit may be promoted, distributed, or sold as a unit for performing a method of the present invention.

The kit can comprise a labeled compound or agent capable of detecting a marker protein or nucleic acid in a sample and means for determining the level of the marker protein or marker nucleic acid in the sample (e.g., an antibody which binds the protein or fragment thereof, or an oligonucleotide probe which binds to DNA or mRNA encoding the protein). Kits can also include instructions for interpreting the results obtained using the kit.

For antibody-based kits, the kit can comprise, for example: (1) a first antibody (e.g., attached to a solid support) which binds to a marker protein; and, optionally, (2) a second, different antibody which binds to either the marker protein or the first antibody and which is optionally conjugated to a detectable label.

For oligonucleotide-based kits, the kit can comprise, for example: (1) an oligonucleotide, e.g., a detectably labeled oligonucleotide, which hybridizes to a nucleic acid marker and/or (2) a pair of primers useful for amplifying a marker nucleic acid molecule. The kit can also comprise, e.g., one or more of the following: a buffering agent, a preservative, or a protein stabilizing agent. The kit can further comprise one or more components for use in detecting the detectable label (e.g., an enzyme or a substrate).

The kit can also contain a control sample or a series of control samples which can be assayed and compared to the test sample. Each component of the kit can be enclosed within an individual container and all of the various containers can be within a single package, along with instructions for interpreting the results of the assays performed using the kit.

It will be appreciated that the kits of the present invention may also include antibodies or oligonucleotide probes which bind to known cancer markers for example known breast cancer markers.

The kit of the invention may optionally comprise additional components useful for performing a method of the invention. By way of example, the kit may comprise fluids (e.g., SSC buffer) suitable for annealing complementary nucleic acids or for binding an antibody with a protein with which it specifically binds, one or more sample compartments, an instructional material which describes performance of a method of the invention, and the like.

In one embodiment, the kit may comprise a microarray, e.g. an oligonucleotide microarray or an antibody microarray. The microarray may be capable of being used to determine the level of one, two, three or four of CDC2, CDC6, DNA2L and CKS2 and optionally also ER-β.

In one embodiment the kit comprises software, for example software for predicting prognostic outcome or for selecting patient treatment. Such software might include instructions for the computer system's processor to receive data structures that include the level of expression of one, two, three or four of the markers of the invention (and optionally one or more further markers) in a sample (e.g. in a breast cancer tumor sample obtained from a breast cancer patient) and optionally also clinical information about the patient, e.g. the patient's age, stage of breast cancer, estrogen receptor status, and lymph node status.

The invention additionally provides a test method of assessing the carcinogenic potential of a test compound. This method may comprise the steps of: maintaining separate aliquots of cancer cells in the presence and absence of a test compound; and comparing expression of a marker of the invention in each of the aliquots. A significantly higher level of expression of the marker in the aliquot maintained in the presence of the compound, relative to that of the aliquot maintained in the absence of the compound, is an indication that the compound possesses carcinogenic potential. Expression of a marker of the invention in an aliquot may also be measured at two or more time points to determine the time required for the compound to effect its carcinogenic potential.

In an alternative embodiment, the level of a marker of the invention may be determined at least two time points in a given sample. In this way, increased or decreased expression of a marker of the invention would indicate the ability of the test compound to promote or inhibit respectively, the cancer.

### EXAMPLES

Reference will now be made in detail to exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

ERα and ERβ bind to the same response element but are associated with different growth effects. In order to assess distinct signatures that can discern the biological differences, the global transcriptional effect of ERβ expression on ERα action was examined.

In order to characterize the effect of ERβ expression on hormone response in ERα-positive breast tumor cells and to decipher the functions of α/β heterodimers, T-47D (ERα+/ERβ−) cells were stably transfected with an inducible ERβ expression construct to generate subline T-47 Dbeta. Using high-density DNA microarrays under conditions that induce ERβ expression and following hormone treatment, the transcriptional effects of the ERα/β coexpression was assessed. It has been shown that ERβ over-expression (induced T47 Dbeta) in this cell line inhibits E2 induced cell proliferation and downregulates the expression and activity of the cell cycle machinery (32).

In the present work, genome-wide expression profiles of genes responsive to ERβ expression and estrogen treatment were generated. Hierarchical clustering of the profiles revealed potential mechanisms of interaction between ERα and ERβ in regulating hormone response, and the potential roles of putative ERβ-modulated genes in the biology of primary breast tumors.

Paradoxically, it was found that induction of ERβ exhibited a highly similar transcriptional response as to the exposure of these ERα-positive cells to estrogen. However, detailed analysis revealed a subset of 14 DNA replication and cell cycle genes specifically down-regulated by ERβ and tightly associated with attenuation of cell proliferation. In order to determine the in vivo relevance of this inhibitory mechanism, the expression characteristics of these genes were examined in 69 ERα-positive tumors from patients who received adjuvant endocrine therapy. Association of the expression characteristics of these genes with ERβ transcript levels, clinical parameters and disease outcome was also assessed. Expression profiles of four ERβ-regulated genes, CDC2, CDC6, CKS2, and DNA2L were significantly associated with ERβ transcript levels, and the directionality is consistent with in vitro observations. Clustering of tumor samples using the expression profiles of the ERβ-associated expression cassette indicated statistically significant association with tumor grade (p=0.00934) but not with lymph node status. This suggests a primary effect of ER-β on tumor growth rather than on the metastatic phenotype.

Kaplan-Meier analysis indicated better disease outcome for the patient group with an expression signature linked to low ERβ expression cassette as compared to the high ERβ cassette group for both disease-free survival (p=0.00165) and disease-specific survival (p=0.0268). These clinical findings were further validated in an independent cohort of 45 ERα-positive tumors and appear to explain the previous survival heterogeneity of this ER positive group.

### Materials and Methods

Cell culture. T-47D cells were cultured in a mixture of DMEM and Hams F12 1:1 supplemented with 5% fetal bovine serum (FBS), penicillin and streptomycim. For experiments using 17β-estradiol, DMEM without phenol red and FBS treated with dextran coated charcoal were used.

Transfection and plasmids. T-47D cells stably transfected with tetracyclin regulated ERβ expression plasmid were generated in two steps: 1) the cells were first transfected with pTet-tTAk (Gibco/Invitrogen, Carlsbad, Calif., USA) modified to contain puromycin resistance using lipofectin according to manufacturer's instructions (Gibco/Invitrogen, Carlsbad, Calif., USA). Selection was performed with 0.5 μg/ml of puromycin in the presense of 1 μg/ml of tetracycline. A good clone showing high level of induction and low basal activity was selected using the pUHC13-3 control plasmid (Gibco/Invitrogen, Carlsbad, Calif., USA). The short form of ERβ encoding 485 amino acids was fused to the flag tag (ERβ 485) and cloned into PBI-EGFP (Clontech/BD Biosciences, Palo Alto, Calif., USA) 2) This construct was then transfected into the previously described inducible clone together with a neomycin resistance plasmid. The selection was performed using 500 μg/ml of G418 (Calbiochem/EMD Biosciences, San Diego, Calif., USA).

Preparation of RNA. Cells were added, to 150 mm plates at a confluency of 40%; after one day, the normal medium was replaced by the medium described above, supplemented with 5% dextran coated charcoal treated FBS (DCCFBS). After 24 h, 10 nM ICI 182,780 was added to the cultures and incubation proceeded for an additional 48 h. For expression of ERβ, tetracycline was removed 12 hours before initiation of treatment with 17β-estradiol. At time 0 h the medium was changed to 0.5% DCCFBS and 10 nM of 17β-estradiol was added. RNA was prepared by adding 10 ml of Trizol. (Invitrogen, Carlsbad, Calif., USA) to each 150 mm plate at different time points after start of treatment and RNA was prepared according to the manufacturer's instructions.

Microarray Analysis. Microarrays were generated by spotting the Compugen (Jamesburg, N.J., USA) 19K human oligo library, made by Sigma-Genosys (The Woodlands, TX, USA), on poly-l-lysine-coated glass slides. 25 μg each of sample total RNA and human universal reference RNA (Stratagene, La Jolla, Calif., USA) were labeled with Cy5-conjugated dUTP and Cy3-conjugated dUTP (PerkinElmer, Boston, Mass., USA); respectively, and hybridized to the arrays using protocols established by the Patrick O. Brown Laboratory (Stanford University, USA) accessible at http://cmgm.stanford.edu/pbrown/protocols/index.html. Array images and data were obtained and processed using the GenePix4000B scanner and GenePix Pro software (Axon Instruments, Union City, Calif., USA). Differentially expressed genes were determined using t-test and fold-difference cut-off at multiple time points and clustered and visualized using the Eisen Cluster and TreeView programs (Eisen et al., 1998). Gene ontology of responsive genes was derived from annotations made by Compugen.

Real-time relative quantification PCR. For validation of microarray data RNA samples from the 30 hour time point were converted to cDNA using the PowerScript™ Reverse Transcriptase (Clontech, CA, USA), following the recommended protocol with 1 μg of total RNA. Quantification of transcript levels was performed using SYBR Green chemistry on the ABI PRISM® 7900HT Sequence Detection System (Applied Biosystems. CA, USA). PCR amplification was carried out in 20 μl of reaction mixture consisting of 50 nM of primer pairs. The following primers were used for PCR reactions: CDC2-CCCAAATGGAAACCAGGAAG (forward) (SEQ ID NO. 12), CGTTTGGCTGGATCATAGAT (reverse) (SEQ ID NO. 13); CDC6-GGAAACCCGTTTGACAAAGG (forward) (SEQ ID NO 14), CGGGTGGGGTGTAAGAGAAGAATT (reverse) (SEQ ID NO. 15); CKS2-CTGATGTCTGAAGAGGAGTG (forward) (SEQ ID NO. 16), GGAAGAGGTCGTCTAAAGAG (reverse) (SEQ ID NO. 17); DNA2L-GGATGGAACTGTTGGTGAAC (forward) (SEQ. ID NO. 18), ATTTAGTGAGGGCACACACC (reverse) (SEQ ID NO. 19); ERβ-TCCATGCGCCTGGCTAAC (forward) (SEQ ID NO. 20), CAGATGTTCCATGCCCTTGTTA (reverse) (SEQ ID NO. 21); β actin-ACCCACACTGTGCCCATCTACGAG (forward) (SEQ ID NO. 22), TCTCCTTAATGTCACGCACGATTTCC (reverse) (SEQ ID NO. 23). 18S rRNA Pre-developed TagMan Assays by Applied Biosystems was used as reference gene.

Patients and tumor specimens. The original patient material consisted of freshly frozen breast tumors from a population-based cohort of 315 women representing 65% of all breast cancers resected in Uppsala County from Jan. 1, 1987 to Dec. 31, 1989 (34, 35). The follow-up RNA expression profiling study was approved by the ethical committee at the Karolinska Institute, Stockholm, Sweden. Affymetrix (Santa Clara, Calif., USA) oligonucleotide microarrays (U133A8d3) were used to determine gene expression profiles in breast tumor samples. After exclusions based on RNA integrity and array quality control, expression profiles of 260 tumors were deemed suitable for further analysis. We selected the 69 ER-positive tumors from patients who underwent adjuvant tamoxifen therapy to assess the role of ERβ-regulated cell cycle and DNA replication genes in breast tumor biology. Clinico-pathological characteristics were derived from the patient records and from routine clinical measurements at the time of diagnosis as described elsewhere (34). Histopathological re-examination and grading according to Elston-Ellis was performed by an experienced breast cancer pathologist without prior knowledge of selected therapies and outcomes.

### Results

### ERβ Expression Elicited Similar Responses as Estrogen Treatment

ERβ expression was induced in the T47 Dbeta subline by tetracycline (TET) withdrawal (see Materials and Methods), and global gene expression profiles in the presence and absence of hormone were determined by human oligonucleotide microarrays (˜19,000 genes, Compugen, Jamesburg, N.J., USA) at 1, 6, 12, 16, and 30 h following induction. Responsive genes were defined as those that exhibited at least a 1.15 fold differential expression between untreated (−E2)-uninduced (+TET) controls and any induced and treated samples in the same direction for three consecutive time points or in four out of the five time points. 3509 genes or 19% (3509/18912) of the genes represented on the arrays were found to be responsive under at least one of the treatment conditions (+E2, +TET; −E2, −TET; or +E2, −TET) as compared to the untreated-uninduced controls. 47% (1662/3509) of the responsive genes were down-regulated as compared to the controls, whereas 53% (1847/3509) were up-regulated. Induction of the ERβ transcript was confirmed as an internal positive control.

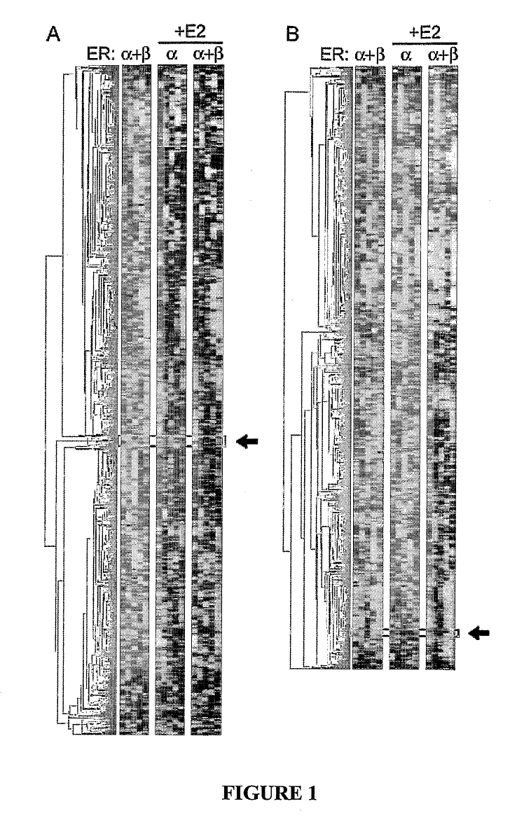

We then examined the expression profiles of responsive genes by hierarchically clustering and visualizing the expression ratios using the Eisen Cluster and TreeView software (33) (see FIG. 1). Surprisingly, the global expression profiles under the three treatment conditions appeared nearly identical for up- (see FIG. 1A) and down-regulated (see FIG. 1B) genes. We assessed the overall similarities between the expression profiles of all genes on the arrays under each of the experimental conditions (uninduced-untreated control, E2 only, ERβ induction only, and E2+ERβ induction) and found a statistically significantly greater correlation (p<1×10⁻⁴ by Monte Carlo simulations) between the three test groups (E2 only, −ERβ induction only, and E2+ERβ induction) as compared to the control samples. These observations suggest that ERβ over-expression in the presence of ERα mimics the activation of ERα by estradiol. Earlier in vitro experiments demonstrated that ERα and ERβ can interact with the same ERE either as homodimers or heterodimers although interaction as heterodimers have not been formally determined (9-11). Our results extend these findings and indicate that one function of ERβ is to augment the basal transcriptional activity of ERα, even in the absence of ligand. These findings are also consistent with previous observations in osteosarcoma cells where ERα and ERβ share the majority of their estrogen responsive genes (31).

### E2 Augments the Disruptive Effects of ERβ Over-Expression on Erα Activity

The significant similarity between E2 and ERβ transcriptional response appears to contradict the confirmed observation that ERβ blocks E2-induced cell proliferation in ERα poSitive cells (32). However, on detailed analysis of the expression profiles, two small clusters of genes emerged which may explain ERβ's antagonistic activity on cell growth. The first cluster, derived from FIG. 1A, contains genes whose expression levels were down-regulated following ERβ expression alone or E2 treatment and ERβ expression but up-regulated following E2 treatment alone (see FIG. 1A, magenta box). Among the genes found within this cluster, proliferating cell nuclear antigen (PCNA) and cell division cycle 6 (CDC6) are both known to be involved in DNA replication and have previously been shown to be estrogen responsive genes (29). The second cluster includes cell division cycle 2 (CDC2) and CDC28 protein kinase regulatory subunit 2 (CKS2), two cell cycle regulatory genes which were also down-regulated by ERβ expression in the presence and absence of E2. The highly specific contrary regulation of a small number of genes, specifically the down-regulation of genes involved in cell cycle regulation and DNA replication, present a plausible mechanism by which ERβ can block E2 induced cell, proliferation. That these genes were down-regulated even in the absence of E2 treatment in serum and estrogen-starved cells indicates that these transcriptional events are causative mechanisms rather than a reflection of cell proliferation inhibition by ERβ. These data support the notion that though the overall expression profile of ERβ/ERα expressing cells resembles that of ligand activated ERα, ERβ specifically alters the expression of a small number of key cell cycle associated genes that can explain its growth inhibitory effects.

E2 Augments the Disruptive Effects of ERβ Over-Expression on ERα Activity

In the absence of hormone, ERβ over-expression appeared to mimic E2 treatment in T-47D cells. The discrepancies that were observed, however, suggest that ERβ can indeed have targeted antagonistic effects on ERα function, specifically on the expression of genes promoting cell proliferation. We extended this analysis to examine the detailed effects of ERβ expression in the presence of the E2 ligand, by directly comparing gene expression levels between the two hormone treated sample groups: induced ERβ expression (−TET)+E2 vs. +E2 alone (+TET). Again, differentially expressed genes were defined as those that have a 1.15 fold change (E2+ERβ/E2) in the same direction for three consecutive time points or four out of the five time points. We posited that the addition of the ligand for ERα and ERβ might augment the differences between the two activated receptors.

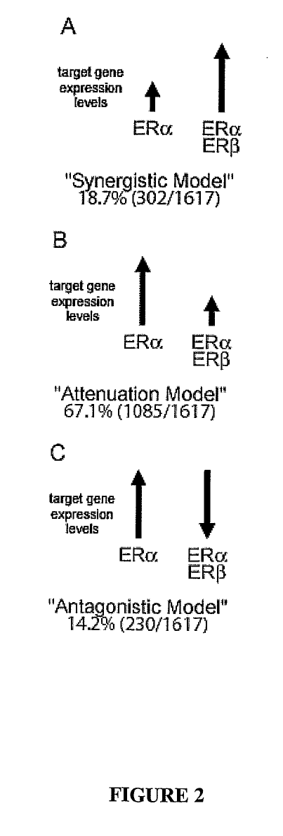

As expected, in contrast to the previous results, we found that the addition of E2 to cells coexpressing ERβ and ERα more obviously perturbed the E2+ERα expression cassette in T-47D cells (N=1617 genes with 893 genes upregulated as compared 729 genes downregulated). From this expression data, we distinguished three models of interaction between ERα and ERβ when liganded with E2: synergistic, attenuation, and antagonistic models (see FIG. 2).

In the synergistic model, ERβ augments the ERα response to E2 (FIG. 2A). 18.7% (302/1617) of the differentially expressed genes exhibited greater responses when ERβ was expressed in the presence of E2 as compared to E2 treatment alone (changes in the same direction, but greater magnitude with ERβ see FIG. 2A). In 67.1% (1085/1617) of the genes, expression of ERβ resulted in the attenuation (changes in the same direction, but decrease in magnitude with ERβ) of the transcriptional response (see FIG. 2B). ERβ expression antagonized (changes in opposite directions) E2 response in 14.2% (230/1617) of differentially expressed genes (see FIG. 2C).

### ERβ Inhibits the Cell Proliferative Effects of Estrogen and Erα by Specifically Blocking the Expression of Hormone Responsive Cell Cycle and DNA Replication Genes

We assessed in an unbiased fashion, whether the genes that were perturbed could be assigned to functional categories by gene ontology and whether genes of a specific function in cell proliferation were statistically significantly enriched when compared to the frequency of these genes represented on the microarray (gene ontology annotated by Compugen, Jamesburg, N.J., USA; see FIG. 2 and summarized in Table 2).

TABLE 2
Statistics of key gene ontology categories

| | | |
|---|---|---|
| Gene Function | % Total | X² p-Value |

E2 + ERβ UP-REGULATED

| | | | |
|---|---|---|---|
| Apoptosis | 1.1% | (10/893) | 0.18590 |
| Cell Cycle | 1.5% | (13/893) | 0.82478 |
| Cell Proliferation | 0.4% | (4/893) | 0.41984 |
| DNA Replication | 0.4% | (4/893) | 0.80576 |

E2 + ERβ DOWN-REGULATED

| | | | |
|---|---|---|---|
| Apoptosis | 0.4% | (3/728) | 0.31923 |
| Cell Cycle | 2.6% | (19/728) | 0.02471 |
| Cell Proliferation | 0.3% | (2/728) | 0.91690 |
| DNA Replication | 1.4% | (10/728) | 0.00175 |

ALL GENES ON ARRAY

| | | | |
|---|---|---|---|
| Apoptosis | 0.7% | (138/18912) | — |
| Cell Cycle | 1.5% | (293/18912) | — |
| Cell Proliferation | 0.3% | (56/18912) | — |
| DNA Replication | 0.5% | (96/18912) | — |

We first observed that ERβ in the presence of E2 did not significantly up-regulate any specific functional class of genes. However, in the down-regulated cassette, we observed 1.7-fold (2.6% vs. 1.5%) enrichment in the frequency of cell cycle genes (chi-square p=0.0241) and a 2.8-fold (1.4% vs. 0.5%) enrichment in DNA replication genes (chi-square p=0.00175). When we divided the 728 down-regulated genes further into the three different models of interaction described in FIG. 2, we found that the cell cycle genes were overrepresented (5.6-fold; chi-square p=0.00077) in the group described by the antagonistic model; i.e., ERβ reversed the induction of cell cycle genes caused by the E2 ligand on ERα. DNA replication genes were enriched 3.2-fold (chi-square p=0.0008) in the group that showed attenuation by ERβ of the E2 mediated transcriptional induction (i.e., the attenuation model, see Table 4. Consistent with the earlier analysis, no functional gene group surveyed was augmented by ERβ. Thus, it appears that ERβ can specifically regulate the proliferative effects of E2 and ERα by reducing the expression levels of components of the cell cycle and DNA replication machinery.

TABLE 3
Statistics of key gene ontology categories by mechanism of regulation
in 728 E2 + ERβ down-regulated genes

| | | |
|---|---|---|
| Gene Function | % Total | X² p-Value |

“SYNERGISTIC”

| | | | |
|---|---|---|---|
| Apoptosis | 0% | (0/129) | 0.33018 |
| Cell Cycle | 0.8% | (1/129) | 0.47730 |
| Cell Proliferation | 0% | (0/129) | 0.53595 |
| DNA Replication | 0.8% | (1/129) | 0.67051 |

“ATTENUATION”

| | | | |
|---|---|---|---|
| Apoptosis | 0.4% | (2/492) | 0.40303 |
| Cell Cycle | 1.8% | (9/492) | 0.11725 |
| Cell Proliferation | 0.2% | (1/492) | 0.70713 |
| DNA Replication | 1.6% | (8/492) | 0.00080 |

“ANTAGONISTIC”

| | | | |
|---|---|---|---|
| Apoptosis | 0.9% | (1/107) | 0.80404 |
| Cell Cycle | 8.4% | (6/107) | 0.00077 |
| Cell Proliferation | 0.9% | (1/107) | 0.22828 |
| DNA Replication | 0.9% | (1/107) | 0.53640 |

ALL GENES ON ARRAY

| | | | |
|---|---|---|---|
| Apoptosis | 0.7% | (138/18912) | — |
| Cell Cycle | 1.5% | (293/18912) | — |
| Cell Proliferation | 0.3% | (56/18912) | — |
| DNA Replication | 0.5% | (96/18912) | — |

Expression Profiles of ERβ Antagonized Cell Cycle Genes are Associated with ERβ Transcript Levels and Disease Outcome in ER+ Breast Cancer Patients

We sought to address whether this in vitro effect of ERβ on proliferation could explain some of the heterogeneity in biological behavior of ERα-positive breast cancer. We posited that higher ERβ levels would be correlated with lower expression of these specific DNA replication and cell cycle genes and, in turn, would be associated with better clinical outcome. To test this hypothesis, we examined ERβ transcript levels in primary breast cancers with the expression profiles of EAβ-regulated cell cycle genes identified in our in vitro analysis, and their association with clinical outcome.

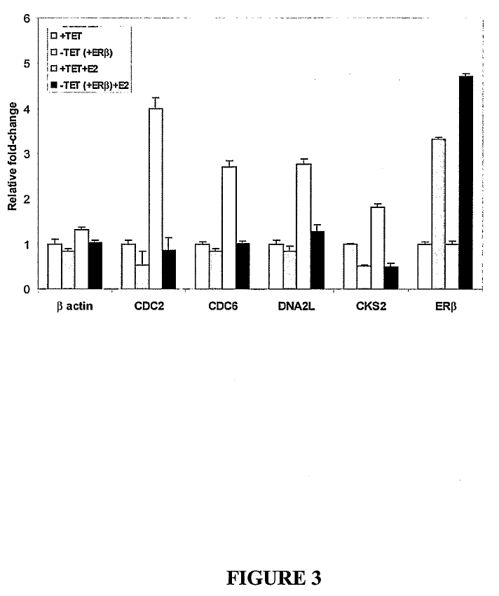

To mimic our in vitro system, we specifically examined microarray expression data from 69 ERα-positive breast tumors from patients belonging to a previously described cohort (34, 35) from Uppsala, Sweden, who had received adjuvant tamoxifen therapy following surgery (see Materials and Methods). We extracted the expression data of the 6 cell cycle and 8 DNA replication genes (see Table 3) which were down-regulated by ERβ in the presence of E2 and classified the tumors base on their expression profiles. Two of the eight DNA replication genes were not found on the arrays used for the tumor studies so only 12 genes in total were available for analysis. We first assessed whether ERβ expression was associated with a decrease in the expression of the ERβ regulated cell cycle genes (i.e. the ERβ cassette). When examined, we found that for the majority of cassette genes (10/12; 83%) there is negative correlation between ERβ transcript levels and the ERβ transcription cassette (see Table 4). This is consistent with our in vitro findings that over-expression of ERβ leads to the down-regulation of the genes in the expression cassette. Four genes in the cassette, CDC2, DNA2L, CDC6, and CKS2, were significantly (student t-test P<0.05) associated with ERβ levels. The estrogen-dependent up-regulation of these genes and the suppression of their expression by ERβ over-expression were further validated by semi-quantitative PCR (see FIG. 3).

TABLE 4
Statistics of association with ERβ expression in 69 breast tumors.

| UniGene ID* | Symbol | Correlation | p-value |
|---|---|---|---|
| Hs.334562 | CDC2 | −0.30798 | 0.00151 |
| Hs.194665 | DNA2L | −0.22919 | 0.01522 |
| Hs.405958 | CDC6 | −0.16138 | 0.02774 |
| Hs.83758 | CKS2 | −0.21564 | 0.04152 |
| Hs.156346 | TOP2A | −0.11536 | 0.07061 |
| Hs.436806 | RAMP | −0.20123 | 0.07442 |
| Hs.436102 | FLJ12484 | 0.05731 | 0.08202 |
| Hs.115474 | RFC3 | −0.19135 | 0.11683 |
| Hs.139226 | RFC2 | −0.09613 | 0.12292 |
| Hs.135665 | GAS2 | 0.05865 | 0.24376 |
| Hs.142179 | INCENP | −0.03967 | 0.29616 |
| Hs.234896 | GMNN | −0.07458 | 0.36950 |
| Hs.22116 | CDC14B** | n/a | n/a |
| n/a | PRO0245** | n/a | n/a |

*UniGene Build 175
**Not found on the arrays

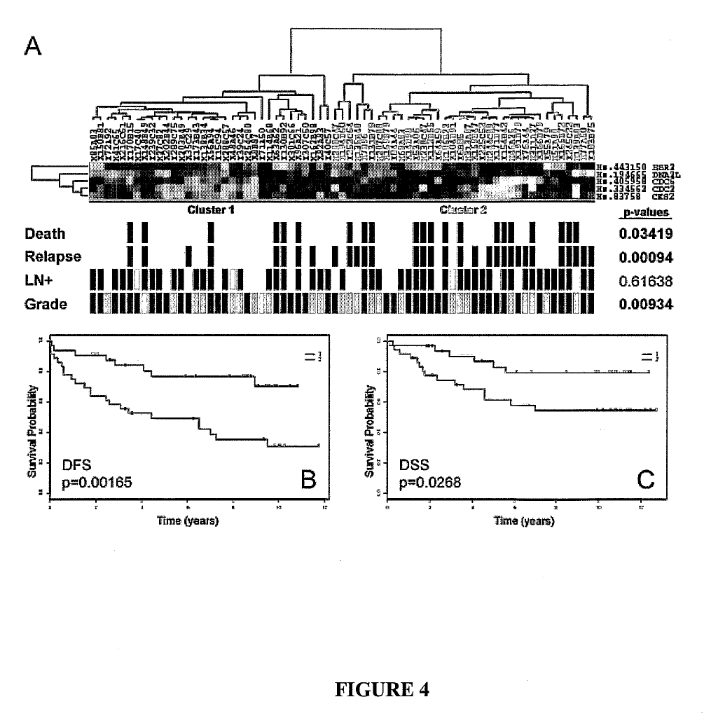

We then used the four ERβ-regulated genes (CDC2, CKS2, CDC6, and DNA2L) with significant correlation with ERβ expression in the tumors as the functional ERβ cassette for subsequent clustering and associations with clinical parameters (see FIG. 4).

Using the expression of the ERβ cassette, the tumor samples were clustered into two groups with statistically significant association with 10-year relapse (chi-square P=0.00094) and death (chi-square P=0.03419), and tumor grade (chi-square P=0.00934).

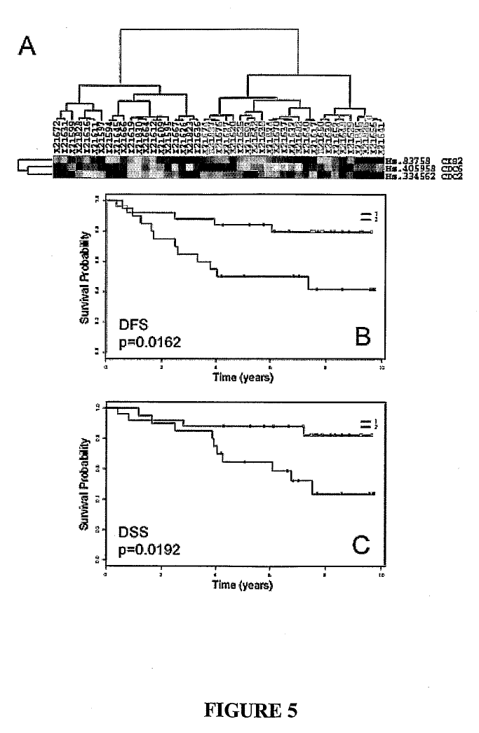

To ascertain the correlation between the molecular signature and disease outcome, we compared the two patient clusters using disease free survival, DFS, (FIG. 4B) and disease specific survival, DSS, (FIG. 4C) as clinical endpoints. Patients with relatively higher levels of ERβ and lower expression of the cassette genes (FIG. 4A, Cluster 1; FIGS. 4B and 4C, black curves) have significantly improved outcomes than the group with lower levels of ERβ and higher levels of the expression cassette (FIG. 4A, Cluster 2; FIG. 4B and 4C, red curves) for both end points, DFS (p=0.0017) and DSS (p=0.0268). As predicted, we did not observe any significant association of the ERβ cassette expression profiles with disease outcome in 37 ERα-negative tumors from the same patient population data not shown). These results Were further validated in an independent dataset (36) of 45 ERα-positive tumors from patients who also underwent endocrine therapy. In this independent dataset of British patients; the ERβ expression cassette profiles also identified high and low ERβ expression cassette patient groups (see FIG. 5A) and to poor and good outcome groups for DFS (FIG. 5B, likelihood ratio P=0.0162) and DSS (FIG. 5C, likelihood ratio P=0.0192) respectively. Our findings indicate that the observed in vitro activity of ERβ on downstream target genes is directly recapitulated in primary breast tumors and has utility in further demarcating prognostic and therapeutic subgroups in the clinical Setting. In addition, these results implicate the selective ERβ transcriptional effects as identified in vitro in defining clinical behavior of human breast cancers.

### Discussion

Original characterization of ERβ function indicated its ability to form homodimers and heterodimers (with ERα) and to bind the same consensus ERE sequences in electrophoretic mobility shift experiments (9). However, ERβ biological effects are considerably different from that of ERα: ERβ expression alone, unlike hormone treatment, was not sufficient to drive cell proliferation (32). In cell lines, over expression of ERβ inhibited E2-driven and ERα-mediated cell proliferation (32). Our finding that over-expression of ERβ in ERα+/ERβ− T-47D cells elicited similar gene expression profiles as hormone treated ERα positive cells was, therefore, surprising. These results suggested that ERα/β heterodimers or ERβ homodimers can increase the basal activity of the ER complex. However, a more detailed genome-wide assessment of transcriptional activity provide strong evidence that over-expression of ERβ in T-47D cells does not fully Mimic E2 treatment, and that there are levels of ordered functional complexity to this interaction: despite the similarity of expression control, a small subset of genes, such as CDC2, CDC6, and PCNA (see Table 2, part 2 and FIG. 3), previously shown to be up-regulated by E2 (29), were down-regulated by ERβ expression. Therefore, in spite of the overall similarities in the ligand-independent functions of ERβ in the presence of ERα and the hormone induced activity of ERα, there are specific regulatory pathways targeted by ERβ that may antagonize key ERα functions (e.g. proliferation).

The initial phenotypic characterization of the cell line showed that over-expression of ERβ blocked E2-driven and ERα-mediated cell proliferation (32). When we directly compared gene expression profiles following ERβ over-expression and hormone treatment to those obtained from cells subjected to hormone treatment only, we now observe the up-regulation of 829 genes and down-regulation of 729 genes, suggesting that ERβ can significantly alter the ERα-mediated transcriptional program mainly in the presence of estrogens. Taken together, our genome-wide analysis has uncovered differential effects of ERβ in a ligand-dependent context: in the absence of ligand, ERβ largely mimics ERα effects except in a small number of genes that affect cell cycle and DNA replication, in the presence of the E2 ligand, ERβ has more apparent antagonistic effects on ERα function. Indeed, statistical analysis indicated a significant enrichment of genes from these two functional categories within the population of genes down-regulated by ERβ in the presence of E2 (see Tables 3 and 4).

One of the difficulties in assessing the biological significance of such associations between in vitro gene expression changes and the cellular response is that a number of genes coordinately expressed as a cassette (e.g., the ERβ cassette) are involved. One-by-one targeting of the genes in the cassette is not only experimentally problematic but also raises the question of relevance given that an entire gene set may need to be altered. We pursued an alternative approach to determining the in vivo relevance of these findings in the engineered cell line by exploring the association of ERβ transcript levels and the key ERβ suppressed cell cycle and DNA replication genes, in disease outcome of breast cancer patients. We hypothesized that if our in vitro findings were biologically important, then its configuration should be captured in clinical tumors and its biological impact should recapitulate that found in cell culture.

To this end, we first found significant association of the expression profiles of four genes, CDC2, CKS2, DNA2L, and CDC6, with ERβ transcript levels in the breast tumors, consistent with the inverse correlation (high ERβ; low cell cycle/DNA replication genes) observed in the cell line model. CDC2 is a cyclin-dependent kinase which regulates G1-S and G2-M phases of the cell cycle; CKS2 encodes the essential regulatory subunit of cyclin-dependent kinases; and CDC6 and DNA2L function in the pre-replication complex formation and unwinding of replicating DNA respectively. Tumor samples were then hierarchically clustered into two distinct groups, using the expression profiles of this four-gene expression cassette and ERβ (see FIG. 4). We found significant association of the patient groups with tumor grade (which we consider a clinical surrogate for cell cycle and DNA replication), and with 10-year relapse and death. Moreover, the patient cluster with high ERβ and low ERβ downstream gene expression have significantly higher probability of disease-free and disease specific survival over time as compared to those with the opposite profiles. These results suggest that the ERβ regulated genes identified in our in vitro studies are also involved in the biology of primary malignant breast tumors and reveal protective effects of ERβ in disease prognosis. That this effect is limited to ERα-positive subgroup is also consistent with our observations that the main ERβ effect is in the presence of ERα and the E2 ligand. Previously, we had identified that ER positive tumors exhibited significant differences detected by expression profiling associated with variances in overall survival (36). Using the same dataset, we assessed the impact of ERβ and its unique downstream expression cassette and found that this minimal gene set alone could account for the majority of the survival differences. This suggests that ERβ status is the major driver for clinical heterogeneity in ER positive tumors.

Our findings are consistent with a recent report by Hopp and colleagues where they found that loW ERβ protein levels are associated with poor disease-free and over-all survival in ER-positive patients treated with adjuvant tamoxifen therapy (37). In contrast, another group reported the lack of association between ERβ transcript-levels and disease outcome following endocrine therapy in a prospective study (38). One possible explanation for the discrepancy between the prospective study and the previous protein level study and the expression cassette data reported here is that protein levels and expression profiles of downstream ERβ regulated genes are a more proximal measure of ERβ activity than the transcript levels alone.

A reasonable question is whether the ERβ downstream genes harbor the transcriptional control elements associated with the estrogen receptors, i.e., EREs. Our assessment of the 5′ regulatory regions of the four key downstream genes CDC2, CKS2, DNA2L, and CDC6 showed no evidence for an acceptable ERE (data not shown). This raises the possibility that either the expression of these genes involve trans-elements such as other transcription factors induced by ERβ, or that ERβ is acting as a co-factor for other transcription factors such as AP-1 (39, 40).

Taken together, our observations also suggest that molecules directly targeting ERα/ERβ interactions or ERβ mimics could be a novel strategy to develop estrogen response modifiers for the management of ERα-positive breast cancers.

It is to be understood that the above description in intended to be, illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

### REFERENCES

1. Ali, S. and Coombes, R. C. Estrogen receptor alpha in human breast cancer: occurence and significance. Journal of Mammary Gland Biology and Neoplasia, 5: 271-281, 2000.
2. Muramatsu, M. and Inoue, S. Estrogen receptors: how do they control reproductive and nonreproductive functions? Biochem Biophys Res Commun, 270:1-10, 2000.
3. Parker, M. G. Structure and function of estrogen receptors. Vitam Horm, 51: 267-287, 1995.
4. Nilsson, S. and Gustafsson; J. A. Estrogen receptor action. Crit Rev Eukaryot Gene Expr, 12: 237-257, 2002.
5. Kuiper, G. G., Enmark, E., Pelto-Huikko, M., Nilsson, S., and Gustafsson, J. A. Cloning of a novel receptor expressed in rat prostate and ovary. Proc Natl Acad Sci USA, 93: 5925-5930, 1996.
6. Kuiper, G. G. Carlsson, B., Grandien, K., Enmark, E., Haggblad, J., Nilsson, S., and Gustafsson, J. A. Comparison of the ligand binding specificity and transcript tissue distribution of estrogen receptors alpha and beta. Endocrinology, 138: 863-870, 1997.
7. Watanabe, T., Inoue, S., Ogawa, S., Ishii, Y., Hiroi, H., Ikeda, K. Orimo, A., and Muramatsu, M. Agonistic effect of tamoxifen is dependent on cell type, ERE-promoter context, and estrogen receptor subtype: functional difference between estrogen receptors alpha and beta. Biochem Biophys Res Commun, 236: 140-145, 1997.
8. Barkhem, T., Carlsson, B., Nilsson, Y., Enmark, E., Gustafsson, J., and Nilsson, S. Differential response of estrogen receptor alpha and estrogen receptor beta to partial estrogen agonists/antagonists. Mol Pharmacol, 54: 105-112, 1998.
9. Pettersson, K., Grandien, K., Kuiper, G. G., and Gustafsson, I. A. Mouse estrogen receptor beta forms estrogen response element-binding heterodimers with estrogen receptor alpha. Mol Endocrinol; 11: 1486-1496, 1997.
10. Cowley, S. M., Hoare, S., Mosselman, S., and Parker, M. G. Estrogen receptors alpha and beta form heterodimers on DNA. J Biol Chem, 272: 19858-19862, 1997.
11. Ogawa, S., Inoue, S., Watanabe, T., Hiroi, H., Orimo, A., Hosoi, T., Ouchi, Y., and Muramatsu, M. The complete primary structure of human estrogen receptor beta (hER beta) and its heterodimerization with ER alpha in vivo and in vitro. Biochem Biophys Res Commun, 243: 122-126, 1998.
12. Hiroi, H., Inoue, S., Watanabe, T., Goto, W., Orimo, A., Momoeda, M., Tsutsumi, O., Taketani, Y., and Muramatsu, M. Differential immunolocalization of estrogen receptor alpha and beta in rat ovary and uterus. J Mol Endocrinol, 22: 37-44, 1999.
13. Sar, M. and Welsch, F. Differential expression of estrogen receptor-beta and estrogen receptor-alpha in the rat ovary. Endocrinology, 140: 963-971, 1999.
14. Sar, M. and Welsch, F. Oestrogen receptor alpha and beta in rat prostate and epididymis. Andrologia, 32: 295-301, 2000.
15. Taylor, A. H. and Al-Azzawi, F. Immunolocalization of oestrogen receptor beta in human tissues. J Mol Endocrinol, 24: 145-155, 2000.
16. Chu, S, and Fuller, P. J. Identification of a splice variant of the rat estrogen receptor beta gene. Mol Cell Endocrinol, 132: 195-199, 1997.
17. Maruyama, K., Endoh, H., Sasaki-Iwaoka, H., Kanou, H., Shimaya, E., Hashimoto, S., Kato, S., and Kawashima, H. A novel isoform of rat estrogen receptor beta with 18 amino acid insertion, in the ligand binding domain as a putative dominant negative regular of estrogen action. Biochem Biophys Res Commun, 246: 142-147, 1998.
18: Petersen, D. N., Tkalcevic, G. T. Koza-Taylor, P. H., Turi, T. G., and Brown, T. A. Identification of estrogen receptor beta2, a functional variant of estrogen receptor beta expressed in normal rat tissues. Endocrinology, 139: 1082-1092, 1998.
19. Hanstein, B., Liu, H., Yancisin, M. C., and Brown, M. Functional analysis of a novel estrogen receptor-beta isoform. Mol Endocrinol, 13: 129-137, 1999.
20. Leygue, E., Dotzlaw, H., Watson, P. H., and Murphy, L. C. Expression of estrogen receptor beta1, beta2, and beta5 messenger. RNAs in human breast tissue. Cancer Res, 59: 1175-1179, 1999.
21. Sanchez, R., Nguyen, D., Rocha, W., White; J. H., and Mader, S. Diversity in the mechanisms of gene regulation by estrogen receptors. Bioessays, 24: 244-254, 2002.
22. Charpentier, A. H., Bednarek, A. K., Daniel, R. L., Hawkins, K. A., Laflin, X. J., Gaddis, S., MacLeod, M. C., and Aldaz, C. M. Effects of estrogen on global gene expression: identification of novel targets of estrogen action. Cancer Res, 60: 5977-5983, 2000.
23. Gruvberger, S., Ringner, M., Chen, Y., Panavally, S., Saal, L. H., Borg, A., Ferno, M., Peterson, C., and Meltzer, P. S. Estrogen receptor status in breast cancer is associated with remarkably distinct gene expression patterns. Cancer Res, 61: 5979-5984, 2001.
24. Soulez, M. and Parker, M. G. Identification of novel oestrogen receptor target genes in human ZR75-1 breast cancer cells by expression profiling. J Mol Endocrinol, 27: 259-274, 2001.
25. Seth, P., Krop, I., Porter, D., and Polyak, K. Novel estrogen and tamoxifen induced genes identified by SAGE (Serial Analysis of Gene Expression). Oncogene, 21: 836-843, 2002.
26. Inoue, A., Yoshida, N., Omoto, Y., Oguchi, S., Yamori, T., Kiyama, R., and Hayashi, S. Development of cDNA microarray for expression profiling of estrogen-responsive genes. J Mol Endocrinol, 29: 175-192, 2002.
27. Peddada, S. D., Lobenhofer, E. K., Li, L., Afshari, C. A., Weinberg, C. R., and Umbach, D. M. Gene selection and clustering for time-course and dose-response microarray experiments using order-restricted inference. Bioinformatics, 19: 834-841, 2003.
28. Lin C Y, Strom A, Vega V B, Li Kong S, Li Yeo A, Thomsen J S, et al. Discovery of estrogen receptor alpha target genes and response elements in breast tumor cells. Genome Biol 2004; 5:R66.
29. Frasor J, Danes J M, Komm B, Chang K C, Lyttle C R, Katzenellenbogen B S. Profiling of estrogen up- and down-regulated gene expression in human breast cancer cells: insights into gene networks and pathways underlying estrogenic control of proliferation and cell phenotype. Endocrinology 2003; 144:4562-74.
30. Lindberg. M K, Moverare S, Skrtic S, Gao H, Dahlman-Wright K, Gustafsson J Å, et Estrogen Receptor (ER)-beta Reduces ERalpha-Regulated Gene Transcription, Supporting a “Ying Yang” Relationship between ERalpha and ERbeta in Mice. Mol Endocrinol 2003; 17:203-8.
31. Stossi F, Barnett D H, Frasor J, Komm B, Lyttle C R, Katzenellenbogen B S. Transcriptional Profiling of Estrogen-Regulated Gene Expression via Estrogen Receptor {alpha} or Estrogen Receptor {beta} in Human Osteosarcoma Cells: Distinct and Common Target Genes for These Receptors**. Endocrinology 2004.
32. Strom A, Hartman J, Foster J S, Kietz S, Wimalasena J, Gustafsson J Å. Estrogen receptor beta inhibits 17beta-estradiol-stimulated proliferation of the breast cancer cell line T47D. Proc Natl Acad Sci USA 2004; 101:1566-71.
33. Eisen M B, Spellman P T, Brown P O, Botstein D. Cluster analysis and display of genome-wide expression patterns. Proc Natl Acad Sci USA 1998; 95:14863-8.
34: Bergh J, Norberg T, Sjogren S, Lindgren A, Holmberg L. Complete sequencing of the p53 gene provides prognostic information in breast cancer patients, particularly in relation to adjuvant systemic therapy and radiotherapy. Nat Med 1995; 1:1029-34.
35. Sjogren S, Inganas M, Norberg T, Lindgren A, Nordgren H, Holmberg L, et al. The p53 gene in breast cancer: prognostic value of complementary DNA sequencing versus immunohistochemistry. J Natl Cancer Inst 1996; 88:173-82.
36. Sotiriou C, Neo S Y, McShane L M, Korn E L, Long P M, Jazaeri A, et al. Breast cancer classification and prognosis based on gene expression profiles from a population-based study. Proc Natl Acad Sci USA 2003; 100:10393-8.
37. Hopp T A, Weiss E L, Parra I S, Cui Y, Osborne C K, Fuqua S A. Low levels of estrogen receptor beta protein predict resistance to tamoxifen therapy in breast cancer. Clin Cancer Res 2004; 10:7490-9.
38. Cappelletti V, Celio L, Bajetta E, Allevi Longarini R, Miodini P, et al. Prospective evaluation of estrogen receptor-beta in predicting response to neoadjuvant antiestrogen therapy in elderly breast cancer patients. Endocr Relat Cancer 2004; 11:761-70.
39. Kushner P J, Agard D A; Greene G L, Scanlan T S, Shiau A K, Uht R M, et al. Estrogen receptor pathways to AP-1. J Steroid Biochem Mol Biol 2000; 74:311-7.
40. Paech K, Webb P, Kuiper G G, Nilsson S, Gustafsson J Å, Kushner P J, et al. Differential ligand activation of estrogen receptors ERalpha and ERbeta at AP1 sites. Science 1997; 277:1508-10.

What is claimed is:

1. A method of assessing ER-β function, the method comprising:
(i) determining the level of at least one marker selected from the group consisting of CDC2, CDC6, DNA2L and CKS2; and
(ii) using the level of the at least one said marker as an indication of ER-β function.

2. A method for cancer prognosis, the method comprising:
(i) determining the level of ER-β in an ER-α positive sample of a patient; and
(ii) using the level of ER-β in making a disease prognosis for the patient.

3. A method for cancer prognosis according to claim 2, wherein the level of ER-β and at least one marker selected from the group consisting of CDC2, CDC6, DNA2L and CKS2 is determined in the ER-α positive sample of a patient.

4. A method of selecting a treatment for a cancer patient, the method comprising:
(i) determining the level of ER-β in an ER-α positive sample of the patient; and
(ii) using the level of ER-β in selecting an appropriate treatment for the patient.

5. A method of selecting a treatment for a cancer patient according to claim 4, wherein the level of ER-β and at least one marker selected from the group consisting of CDC2, CDC6, DNA2L and CKS2 is determined in the ER-α positive sample of the patient.

6. A method of characterizing a cancerous cell or a cell suspected of being cancerous, the method comprising:
(i) determining the level of ER-β; and
(ii) using the level of ER-β in characterizing the cell(s);
wherein the cell(s) are ER-α positive.

7. A method of characterizing a cancerous cell or a cell suspected of being cancerous according to claim 6, wherein the level of ER-β and at least one marker selected from the group consisting of CDC2, CDC6, DNA2L and CKS2 is determined in the ER-α positive cell(s).

8. A method according to any one of claims 1 to 7 wherein the method comprises determining the level of at least two or three markers selected from the group consisting of CDC2, CDC6, DNA2L and CKS2.

9. A method according to any one of claims 1 to 7 wherein the method comprises determining the level of CDC2, CDC6, DNA2L and CKS2.

10. A method according to any one of claims 2 to 7 wherein the cancer is selected from the group consisting of breast cancer, prostate cancer and colon cancer.

11. A method according to any one of claims 1 to 7 wherein the method further comprises determining the level of a further marker wherein the further marker is not selected from the group consisting of CDC2, CDC6, DNA2L and CKS2.

12. A method according to claim 11 wherein the further marker is selected from Table 4.

13. A method according to any one of claims 2 to 7 wherein the cancer has been treated with adjuvant endocrine therapy.

14. A method according to claim 1 wherein the method is used for: (i) assessing treatment efficacy or cancer progression or regression; (ii) identifying a compound useful for the treatment of cancer; or (iii) assessing the carcinogenic potential of a test compound.

15. An array for assessing ER-β function comprising probes, wherein the probes are configured to determine the level of at least one marker selected from the group consisting of CDC2, CDC6, DNA2L and CKS2, and wherein the determined level of the at least one marker is used to assess ER-β function.

16. An array according to claim 15 wherein the array comprises probes for determining the level of CDC2, CDC6, DNA2L and CKS2.

17. An array according to claim 15 wherein the probes are bound to nucleic acids or proteins derived from a sample which is suspected of being cancerous or which is known to be cancerous.

18. A kit for assessing ER-β function comprising a reagent capable of determining the level of at least one marker selected from the group consisting of CDC2, CDC6, DNA2L and CKS2, and wherein the determined level of the at least one marker is used to assess ER-β function.

19. A kit according to claim 18 comprising reagents for determining the level of CDC2, CDC6, DNA2L and CKS2.
